(19) United States
(12) Patent Application Publication
(10) Pub. No.: US 2025/0152702 A1
(43) Pub. Date: May 15, 2025

(54) EPICHAPEROME INHIBITION TO IMPROVE T CELL RESPONSES

(71) Applicants: Monica L. Guzman, Ithaca, NY (US); Gail J. Roboz, New York, NY (US); Gabriela Chiosis, New York, NY (US); Olivier Boyer, Rouen (FR); Jérémie Martinet, Rouen (FR)

(72) Inventors: Monica L. Guzman, Ithaca, NY (US); Gail J. Roboz, New York, NY (US); Gabriela Chiosis, New York, NY (US); Olivier Boyer, Rouen (FR); Jérémie Martinet, Rouen (FR)

(21) Appl. No.: 18/949,936

(22) Filed: Nov. 15, 2024

Related U.S. Application Data
(60) Provisional application No. 63/599,371, filed on Nov. 15, 2023.

(51) Int. Cl. A61K 39/39; A61K 31/52; A61K 35/17; A61K 39/00; A61K 40/11; A61K 40/32; A61P 35/00; A61P 37/04

(52) U.S. Cl. CPC A61K 39/39; A61K 31/52; A61K 35/17; A61K 40/11; A61K 40/32; A61P 35/00; A61P 37/04; A61K 2039/572

(57) ABSTRACT

Compositions and methods are described herein that are useful for improving the cytotoxicity of T cells and thus, have use in improving the effectiveness of cellular therapies and immune therapies, including, but not limited to, stem cell transplantation and CAR T cell therapy. The compositions and methods can also be used as an adjuvant for infectious disease vaccines to reduce or prevent infections in a human.

### PRIORITY

This application claims the benefit of the filing date of U.S. provisional application No. 63/599,371, filed on Nov. 15, 2023, the disclosures of which is incorporated by reference herein in its entirety.

### STATEMENT REGARDING FEDERALLY FUNDED RESEARCH

This invention was made with government support under grant numbers 1 R01 CA234478-01 and 2 R01 CA098571-16A1 awarded by the National Cancer Institute, the government has certain rights in this invention.

### BACKGROUND

Currently, immunotherapies are being used to treat different cancers as re-engaging the immune system may be a strategy to maintain long lasting remissions. Approaches are being used with the goal of improving immune therapy response, that includes checkpoint inhibitors, such as atezolizumab, avelumab, durvalumab. Despite the success of some immunotherapies, many patients relapse.

### SUMMARY

Provided herein are methods of improving cytotoxicity of antigen-specific T cells in an animal, comprising administering an antigen to the animal to prime a T-cell response; and administering an epichaperone disrupter to the animal during a priming phase with the antigen. Some aspects provide administering the epichaperome disrupter to the animal concurrently with the antigen so as to increase the number of IFNγ-secreting T cells compared to administering the antigen without the epichaperone disrupter. In some embodiments, the animal is a human. In some embodiments, the epichaperome disrupter is one or more compounds of Formula 1, 1A, 1B, 1C, 2, 3, 4, 5, 6, II, and/or III.

Another embodiment provides methods of improving cytotoxicity of antigen-specific T cells in an animal, comprising administering an immunotherapy to the animal; and administering an epichaperome disruptor to the animal with the immunotherapy. In some embodiments, the immunotherapy comprises a cellular immunotherapy. In some embodiments, the cellular immunotherapy comprises chimeric antigen receptor (CAR)-T cell therapy or stem cell transplantation. In some embodiments, the epichaperome disruptor is administered to the animal in an amount effective for improving the antitumor efficacy of the CAR-T cells. In some embodiment, the epichaperome disruptor is administered to the animal in an amount effective for increasing GvL and decreasing GvHD. In some embodiments, the animal is a human. In some embodiments, the epichaperome disrupter is one or more compounds of Formula 1, 1A, 1B, 1C, 2, 3, 4, 5, 6, II, and/or III.

Also provided herein are antitumor vaccines for treating cancer in a human, the antitumor vaccine comprising: an adjuvant comprising an epichaperome disruptor; and a cellular immunotherapy. In some embodiments, the cellular immunotherapy comprises chimeric antigen receptor (CAR)-T cells (CAR-T cells) derived from the human's T-cells and modified to bind one or more antigens on a surface of cancer cells in the human. In some embodiments, the cellular immunotherapy comprises stem cell transplantation. In some embodiments further comprise an antigen. In some embodiments, the epichaperome disrupter is one or more compounds of Formula 1, 1A, 1B, 1C, 2, 3, 4, 5, 6, II, and/or III.

Provided here are vaccines for reducing or preventing infection in a human, the vaccine comprising: an adjuvant comprising an epichaperome disruptor; and an antigenic preparation or an an antigen-encoding RNA preparation. In some embodiments, the epichaperome disrupter is one or more compounds of Formula 1, 1A, 1B, 1C, 2, 3, 4, 5, 6, II, and/or III.

In some embodiments, the epichaperome disrupter is PU-H71 (8-[(6-iodo-1,3-benzodioxol-5-yl)sulfanyl]-9-[3-(propan-2-ylamino)propyl]purin-6-amine; pubchem.ncbi.nlm.nih.gov/compound/9549213; incorporated herein by reference). In some embodiments, the epichaperome disrupter is not PU-H71. In some embodiments, the epichaperome disrupter is a compound as disclosed in WO2011044394 (incorporated herein by reference). In some embodiments, the epichaperome disrupter is a compound as disclosed in WO2012138894 (incorporated herein by reference). In some embodiments, the epichaperome disrupter is a compound as disclosed in WO2012138896 (incorporated herein by reference).

### BRIEF DESCRIPTION OF THE DRAWINGS

Exemplary embodiments are illustrated in referenced figures. It is intended that the embodiments and figures disclosed herein are to be considered illustrative rather than restrictive.

FIGS. 1A-1C. Schematic representation of investigation. (A) Determine the role of the epichaperome in T cell activation; data indicates that the epichaperome plays a role in activated T cells that has not been previously described. Biochemical assays determine the kinetics of the epichaperome formation, and the proteins being recruited to the scaffold. The effect of the epichaperome inhibition on T cell phenotype and function are also determined. (B) Assess the combination of PU-H71 with CAR T cell therapy. It was observed that epichaperome disruption using PU-H71 can significantly improve T cell cytotoxic activity when added to co-cultures of CAR T cells targeting CD19 (CART19) and CD19⁺ leukemia cells. The combination of PU-H71 with CAR T cells targeting CD19 against CD19⁺ malignancies was tested using in vitro and in vivo assays. (C) Determine the impact of PU-H71 in GvL and GvHD. Data shows that epichaperome disruption can enhance anti-tumor immune responses and that it does not exacerbate graft vs host disease (GvHD).

FIGS. 2A-2D. Epichaperomes are formed in activated T cells and their disruption impacts T cell activation phenotype but not survival. (A) Resting T cells from healthy donors were assayed for the abundance of epichaperomes by flow cytometry. (B) Purified T cells activated with anti-CD3/CD28 beads were tested for changes in epichaperome abundance 20 h after activation and compared with resting T cells and MV4-11 as a positive control. n=3, One-way ANOVA ** p<0.002, *** p<0.0002 (C, D) T cells were treated with 1 μM PU-H71 upon activation to assess the effect of epichaperome disruption on survival (C) and cell surface phenotype (CD4, CD8, CD25, CD69, HLA-DR) n=3 *p<0.03 two-tailed T-test (D).

FIGS. 3A-3H. Disruption of the epichaperome using PU-H71 improves OVA-specific T cells response and anti-tumor immune response in a mouse model. (A) C57BL/6 (B6) mice were injected intraperitoneally (i.p.) with PU-H71 (75 mg/kg) n=8 or PBS n=8 on days 0, 3 and 7. On day 1, all mice were immunized subcutaneously (s.c.) with 100 μg OVA emulsified in incomplete Freund's adjuvant (IFA), experimental controls shown in schema. Peripheral blood (PB) was collected on days 0, 5 and 9, and the spleen was collected at the end of the experiment, on day 9. (B, C) T cell immunization against OVA was assessed by measuring IFN□ release from T cells after exposure with OVA using Elispot assay. Two-tailed T test (D) Anti-OVA IgG in serum was assessed by ELISA. (E) B6 mice were immunized with OVA treated with PU-H71 (75 mg/kg) n=8 or PBS n=8 on days 11, 15 and 18. PB and spleen were collected at the end of the experiment, on day 20. (F) T cell immunization against OVA was assessed by measuring IFN□ by Elispot assay. (G) B6 mice were immunized with OVA/IFA, or with OVA alone, or with PBS as a control, mice were treated with PU-H71 (75 mg/kg) n=8 or PBS n=8 at the indicated days. On day 7 after immunization, mice challenged with OVA-expressing EG.7 tumor cells (H) tumor volume was assessed every 2-3 days using calipers. p values shown for day 13 assessment by one-way ANOVA.

FIG. 4. Epichaperome in tumor immune evasion. The epichaperome immunomodulatory protein networks include several immunosuppressive pathways. Highlighted in red is the ‘Cytokine signaling’, as an example.

FIGS. 5A-5C. In vitro assessment of epichaperome abundance in CAR T cells and its effect on their function. (A) Epichaperome formation in CAR T cells will be evaluated by flow cytometry after activation with anti-CD3/CD28 beads or 3T3-CD19 (CAR activation) or 3T3-CD22 (negative control). (B) CART19 will be co-cultured with 3T3-CD19 or 3T3-CD22 (control) for 24 h to evaluate their cytotoxicity by flow cytometry. PU-H71 (1 μM) or PBS will be added at different time points. (C) CART19/4-1BB will be co-cultured with anti-CD3/CD28 beads or 3T3-CD19 or 3T3-CD22 in the presence or absence of PU-H71 to characterize the changes in T cell subpopulations using epichaperomics and sc/CITEseq.

FIGS. 6A-6B. The data provides in vitro co-cultures of CART cells targeting human CD19, tested against EL4 cells (mouse lymphoma cells) expressing either human CD19 or human CD22 (mouse cell line used to decrease any allogeneic effect that one may see with the CART cells). Two different conditions were tested. In red the effect demonstrates when the epichaperome inhibitor (PU) is added 1 hour before the CART cells and in green the effect when PU is added 2 hours after the CART cells is shown. (A) The specific activity against the target (CD19); (B) the the control testing against CD22.

FIGS. 7A-7C. Epichaperome disruption improves CAR T cell cytotoxicity. (A) Schematic representation. CART19/4-1BB or control T cells were cultured with NALM6 (CD19⁺ B-ALL cell line) for 18 h at a E:T ratio of 1:1. (B) Cytotoxicity was evaluated by flow cytometry and represented as relative to the number of dead cells in the control cells (no effector cells). Experiment performed in triplicate, Error bar represents the SEM, significance assessed using ANOVA test. (C) In vivo testing.

FIGS. 8A-8D. Epichaperome disruption does not aggravate GvHD. (A) Experimental design of the pilot study. Lethally irradiated (10 Gy) B6×C3 F1 mice were transplanted with C57BL/6 mice BM cells followed with an i.v. injection of T cells from splenocytes from the same donor. Mice were treated i.p. with PU-H71 as indicated. (B) Survival and (C) GvHD clinical score were evaluated. (D) Representative immunohistochemistry from the indicated organs and treatment are shown.

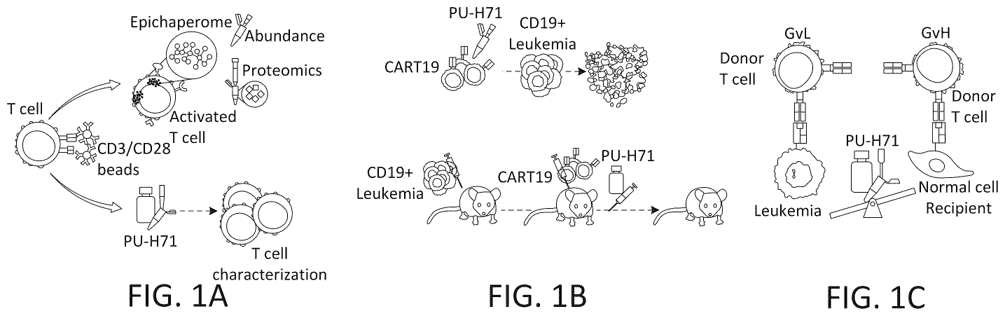

### DESCRIPTION OF THE INVENTION

Compositions and methods are described herein that are useful for improving the cytotoxicity of T cells and thus, have use in improving the effectiveness of cellular therapies and immune therapies, including, but not limited to, stem cell transplantation and CAR T cell therapy. The compositions and methods can also be used as an adjuvant for infectious disease vaccines to reduce or prevent infections in a human.

The effects of an epichaperome disrupter, PU-H71, were tested on C57BL/6 mice immune responses to ovalbumin (OVA) immunization. OVA-specific T- and B-cell responses were evaluated by ELISPOT and ELISA, respectively. When administered at the time of priming, PU-H71 significantly increased the number of IFNγ-secreting T cells as compared to controls (median 126 vs 42 per million of cells, p=0.0002; n=8), without an impact on T cell phenotype or anti-OVA antibody levels. In contrast, PU-H71 had no effect when administered once the OVA-specific T cell response was ongoing.

The effect of PU-H71 on antitumor immune responses in OVA-primed mice challenged with E.G7 (OVA+) or parental EL4 lymphoma cell lines (OVAneg) was tested. PU-H71 improved in vivo anti-tumor immune responses in OVA-primed mice challenged with E.G7 but not EL4 cells.

Since PU-H71 augmented early but not established T cell responses, it was next evaluated whether it would have an effect on a graft versus host disease (GvHD) murine model. Lethally irradiated B6C3F1 mice received bone marrow and splenocytes from C57BL/6 mice and were treated with PU-H71 or vehicle at the first signs of GvHD (D11). GvHD clinical scores and histological analysis (colon, small intestine, liver and skin) were assessed. PU-H71 did not aggravate GvHD at both clinical and histological levels. Therefore, PU-H71 augments antigen-specific mouse T cell responses by acting during priming.

To better understand its effect on human T cells and its potential adjuvant effect during cellular therapy, in vitro and in vivo chimeric antigen receptor (CAR)-T cell (CAR-T cells) phenotype and function in the presence of PU-H71 was tested. T cells were purified from healthy donor PBMCs and were transduced with a CD19.4-1Bb.3z lentiviral vector to obtain CART19 cells. It was found that epichaperone forms in CART19 cells upon activation with anti-CD3/CD28 beads vs control. In vitro cytotoxicity of CAR-T cells toward NALM6 B-ALL cells was significantly improved (55.39 vs 74.66%, p=0.0356) by PU-H71. Finally, the therapeutic effect of the combination of PU-H71 and CART19 was evaluated in a B-ALL NSG mouse model using NALM6-BLIV cells, with bioluminescence imaging and flow cytometry (CART19 and leukemic cells) monitoring. Results show that PU-H71 administration increases CART19 numbers in the bone marrow and improves their in vivo antitumor efficacy against NALM6.

These results show that PU-H71 augments antigen-specific T cell responses during priming including CAR-T cell cytotoxicity, with little effect on ongoing immune responses including GvHD. The data suggest that PU-H71 improves the in vivo efficiency of CAR-T cells in a B-ALL mouse model. In addition to its previously demonstrated direct anti-tumor effect, PU-H71 also had an adjuvant effect on anti-tumor T cell responses while not aggravating GvHD, which supports its clinical evaluation in the post-transplant setting or in combination with immune therapies like CAR-T cells, showing potential for vaccine adjuvant therapy.

### Definitions

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application.

References in the specification to “one embodiment,” “an embodiment,” etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations. “Plurality” means at least two.

The term “and/or” means any one of the items, any combination of the items, or all of the items with which this term is associated. The phrase “one or more” is readily understood by one of skill in the art, particularly when read in context of its usage. For example, one or more substituents on a phenyl ring refers to one to five, or one to four, for example if the phenyl ring is di-substituted.

As used herein, “or” should be understood to have the same meaning as “and/or” as defined above. For example, when separating a listing of items, “and/or” or “or” shall be interpreted as being inclusive, e.g., the inclusion of at least one, but also including more than one of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including,” “includes,” “having,” “has,” “with,” or variants thereof, are intended to be inclusive similar to the term “comprising.” As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. The term about can also modify the endpoints of a recited range as discuss above in this paragraph. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about.”

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to,” “at least,” “greater than,” “less than,” “more than,” “or more,” and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

The terms “patient,” “individual,” or “subject” are used interchangeably herein, and refer to a mammal, particularly, a human. The patient may have mild, intermediate or severe disease. The patient may be an individual, at risk of developing a disease, in need of treatment or in need of diagnosis based on particular symptoms or family history. In some cases, the terms may refer to treatment in experimental animals, in veterinary application, and in the development of animal models for disease, including, but not limited to, rodents including mice, rats, and hamsters, and primates. Mammals include, but are not limited to, humans, farm animals, sport animals and pets (e.g., companion animals, such as dogs, cats, mini pigs, birds).

As used herein, “health care provider” includes either an individual or an institution that provides preventive, curative, promotional or rehabilitative health care services to a subject, such as a patient. In one embodiment, the data is provided to a health care provider so that they may use it in their diagnosis/treatment of the patient.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art, especially in light of the detailed disclosure provided herein. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

As used in this application, the term “treatment” refers to delaying the onset of symptoms, reducing the severity or delaying the symptomatic progression of cancer, neurodegenerative disease or other condition in the individual. A cure of the disease is not required to fall within the scope of treatment. Further, it will be appreciated that the specific results of these treatment goals will vary from individual to individual, and that some individuals may obtain greater or lesser benefits than the statistical average for a representative population. Thus, treatment refers to administration of composition to an individual in need, with the expectation that they will obtain a therapeutic benefit. The terms “treating,” “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

The term “administering” refers to the act of introducing into the individual the therapeutic compound. In general, any route of administration can be used. Thus, administration by oral, intrathecal, intravenous, intramuscular or parenteral injection is appropriate depending on the nature of the condition to be treated. Administration may also be done to the brain by inhalation because there is a compartment at the upper side of the nose that connects with the brain without having the BBB capillaries. Compounds that cross the blood brain barrier are preferred for this mode of administration, although this characteristic is not strictly required.

A “compound,” as used herein, refers to any type of substance or agent that is commonly considered a drug, or a candidate for use as a drug, as well as combinations and mixtures of the above.

As used herein, the term “pharmaceutically-acceptable carrier” means a chemical composition with which an appropriate compound or derivative can be combined and which, following the combination, can be used to administer the appropriate compound to a subject. “Pharmaceutically acceptable” means physiologically tolerable, for either human or veterinary application. As used herein, “pharmaceutical compositions” include formulations for human and veterinary use.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

The term “therapeutically effective amount” encompasses both the amount of the compound administered and the schedule of administration that on a statistical basis obtains the result of preventing, reducing the severity or delaying the progression of the disease in the individual. As will be appreciated, preferred amounts will vary from compound to compound in order to balance toxicity/tolerance with therapeutic efficacy and the mode of administration. Determination of maximum tolerated dose and of the treatment regime in terms of number and frequency of dosing is a routine part of early clinical evaluation of a compound.

### Epichaperome

Chaperome refers to the ensemble of all cellular molecular chaperone and co-chaperone proteins that assist protein folding of misfolded proteins or folding intermediates in order to ensure native protein folding and function, to antagonize aggregation-related proteotoxicity and ensuing protein loss-of-function or protein misfolding-diseases such as the neurodegenerative diseases Alzheimer's, Huntington's or Parkinson's disease, as well as to safeguard cellular proteostasis and proteome balance.

The term chaperome was first coined in a 2006 publication in Cell by Balch and co-workers on the finding that down-regulation of the Hsp90 co-chaperone Aha1 rescues misfolding of CFTR in cystic fibrosis to describe the overall chaperone folding environment, or the “chaperome”. In 2014, Brehme and co-workers systematically studied the expression dynamics of the full human chaperome comprising ˜300 human chaperones and co-chaperones in human aging brains and in brains of patients with neurodegenerative diseases. Integration with chaperome-wide functional RNA interference (RNAi) perturbation experiments in worm and in human cells led to the identification of a chaperome sub-network that safeguards proteostasis in aging and neurodegenerative diseases.

A comprehensive literature survey reviewed the literature since the release of the human genome sequence in 2000 for systematic studies in small animal model systems and highlighted the power of model systems to unveil those key chaperone modifiers of proteotoxicity out of the large number represented in the wider human chaperome that could inform targets and strategies for therapeutic regulation of chaperone functionality.

In 2016, a Nature article authored by Rodina et al. introduced a novel term, epichaperome, to refer to a network of existent chaperomes that are found in cancer cells. More specifically, a few of the major players of this network include heat-shock protein 90 (Hsp90) and heat-shock cognate protein 70 (Hsc70). As opposed to in normal, healthy cells, where these chaperomes are abundant and functional on their own, in cancer cells, changes in the interactions between chaperomes lead to the formation of a network of chaperomes, co-chaperomes, and related co-factors. What has been found is that this strengthened network amongst chaperomes is a mechanism for survival for cancer cells when adapting to stress including hypoxia and heat. The small molecule Hsp90 inhibitor, PU-H71, has been found to have a preferential binding to Hsp90 when it is in the highly integrated complexed form that is the epichaperome.

### T Cells/CAR T

T cells are one of the important types of white blood cells of the immune system and play a central role in the adaptive immune response. T cells can be distinguished from other lymphocytes by the presence of a T-cell receptor (TCR) on their cell surface.

T cells are born from hematopoietic stem cells, found in the bone marrow. Developing T cells then migrate to the thymus gland to develop (or mature). T cells derive their name from the thymus. After migration to the thymus, the precursor cells mature into several distinct types of T cells. T cell differentiation also continues after they have left the thymus. Groups of specific, differentiated T cell subtypes have a variety of important functions in controlling and shaping the immune response.

One of these functions is immune-mediated cell death, and it is carried out by two major subtypes: CD8+ “killer” (cytotoxic) and CD4+ “helper” T cells. (These are named for the presence of the cell surface proteins CD8 or CD4.) CD8⁺ T cells, also known as “killer T cells”, are cytotoxic—this means that they are able to directly kill virus-infected cells, as well as cancer cells. CD8⁺ T cells are also able to use small signaling proteins, known as cytokines, to recruit other types of cells when mounting an immune response. A different population of T cells, the CD4⁺ T cells, function as “helper cells”. Unlike CD8⁺ killer T cells, the CD4⁺ helper T (T_{H}) cells function by further activating memory B cells and cytotoxic T cells, which leads to a larger immune response. The specific adaptive immune response regulated by the T_{H} cell depends on its subtype (such as T-helper1, T-helper2, T-helper17, regulatory T-cell), which is distinguished by the types of cytokines they secrete.

Regulatory T cells are yet another distinct population of T cells that provide the critical mechanism of tolerance, whereby immune cells are able to distinguish invading cells from “self”. This prevents immune cells from inappropriately reacting against one's own cells, known as an “autoimmune” response. For this reason, these regulatory T cells have also been called “suppressor” T cells. These same regulatory T cells can also be co-opted by cancer cells to prevent the recognition of, and an immune response against, tumor cells.

In biology, chimeric antigen receptors (CARs)—also known as chimeric immunoreceptors, chimeric T cell receptors or artificial T cell receptors—are receptor proteins that have been engineered to give T cells the new ability to target a specific antigen. The receptors are chimeric in that they combine both antigen-binding and T cell activating functions into a single receptor.

CAR T cell therapy uses T cells engineered with CARs to treat cancer. T cells are modified to recognize cancer cells and destroy them. The standard approach is to harvest T cells from patients, genetically alter them, then infuse the resulting CAR T cells into patients to attack their tumors.

CAR T cells can be derived either autologously from T cells in a patient's own blood or allogeneically from those of a donor. Once isolated, these T cells are genetically engineered to express a specific CAR, using a vector derived from an engineered lentivirus such as HIV (see Lentiviral vector in gene therapy). The CAR programs the T cells to target an antigen present on the tumor cell surface. For safety, CAR T cells are engineered to be specific to an antigen that is expressed on a tumor cell but not on healthy cells.

After the modified T cells are infused into a patient, they act as a “living drug” against cancer cells. When they come in contact with their targeted antigen on a cell's surface, T cells bind to it and become activated, then proceed to proliferate and become cytotoxic. CAR T cells destroy cells through several mechanisms, including extensive stimulated cell proliferation, increasing the degree to which they are toxic to other living cells (cytotoxicity), and by causing the increased secretion of factors that can affect other cells such as cytokines, interleukins and growth factors.

The surface of CAR T cells can bear either of two types of co-receptors, CD4 and CD8. These two cell types, called CD4+ and CD8+, respectively, have different and interacting cytotoxic effects. Therapies employing a 1-to-1 ratio of the cell types apparently provide synergistic antitumor effects.

The first step in the production of CAR T-cells is the isolation of T cells from human blood. CAR T-cells may be manufactured either from the patient's own blood, known as an autologous treatment, or from the blood of a healthy donor, known as an allogeneic treatment. The manufacturing process is the same in both cases; only the choice of initial blood donor is different.

First, leukocytes are isolated using a blood cell separator in a process known as leukocyte apheresis. Peripheral blood mononuclear cells (PBMCs) are then separated and collected. The products of leukocyte apheresis are then transferred to a cell-processing center. In the cell processing center, specific T cells are stimulated so that they will actively proliferate and expand to large numbers. To drive their expansion, T cells are typically treated with the cytokine interleukin 2 (IL-2) and anti-CD3 antibodies. Anti-CD3/CD28 antibodies are also used in some protocols.

The expanded T cells are purified and then transduced with a gene encoding the engineered CAR via a retroviral vector, typically either an integrating gammaretrovirus (RV) or a lentiviral (LV) vector. These vectors are very safe in modern times due to a partial deletion of the U3 region. The new gene editing tool CRISPR/Cas9 has recently been used instead of retroviral vectors to integrate the CAR gene into specific sites in the genome.

The patient undergoes lymphodepletion chemotherapy prior to the introduction of the engineered CAR T-cells. The depletion of the number of circulating leukocytes in the patient upregulates the number of cytokines that are produced and reduces competition for resources, which helps to promote the expansion of the engineered CAR T-cells.

As of March 2019, there were around 364 ongoing clinical trials happening globally involving CAR T cells. The majority of those trials target blood cancers: CAR T therapies account for more than half of all trials for hematological malignancies. CD19 continues to be the most popular antigen target, followed by BCMA (commonly expressed in multiple myeloma). In 2016, studies began to explore the viability of other antigens, such as CD20. Trials for solid tumors are less dominated by CAR T, with about half of cell therapy-based trials involving other platforms such as NK cells.

As an example, cancer, including blood cancers, such as lymphoma. Leukemia, and myeloma, and autoimmune diseases can be treated with CAR T therapy.

### GvL/GvHD

GvL is also synonymous with graft vs tumor in non-leukemic disease. As the process is mediated by donor immune cells, GvL frequently coincides with alloreactive toxicity against the donor's epithelial cells, resulting in graft versus host disease (GvHD).

Graft-versus-host disease (GvHD) is a clinical problem after, for example, allogenic hematopoietic cell transplantation (HCT), associated with substantial morbidity and mortality that limits the potential utility of transplantation. Associated with GvHD is the well-recognized phenomenon of the graft-versus-leukemia (GvL) effect that results in reduced risk of disease relapse. GvL effects have been observed after treatment for a broad range of hematological malignancies. Both GvHD and GvL are the results of T cell-effector functions. A major goal of basic science and translational research has been to develop strategies to reduce the risk of GvHD while maintaining or enhancing GvL.

Allogenic hematopoietic cell transplantation (HCT) is an established therapy for a broad range of hematological malignancies, bone marrow failure states, and genetic diseases. After transplantation, donor-derived T cells provide many functions, such as enhancement of engraftment, protection from opportunistic infections, and, in the setting of malignancies, rejection of the underlying disease. However, these same T cells also result in graft-versus-host disease (GvHD) that can range from a mild skin rash to a life-threatening and in some instances life-ending complication. GvHD and the risk of opportunistic infections have limited the potential utility of allogenic HCT that could also be an effective therapy for the treatment of patients with severe autoimmune disorders, induction of organ transplantation tolerance, and perhaps other clinical settings if these problems could be addressed effectively. It has been recognized for several decades that a major benefit of allogenic HCT has been the graft-versus-leukemia (GvL) effect that is a result of donor T cells capable of recognizing residual tumor cells and rejecting these cells, resulting in dramatically reduced risk of relapse. The GvL concept was developed from many lines of evidence, including the increased risk of relapse after syngenic transplantation, the finding that T-cell depletion resulted in reduced risk of GvHD but also increased relapse rates, a decreased relapse rate associated with chronic GvHD, and finally the utility of donor leukocyte infusions (DLIs) for the treatment of patients who suffer a relapse after allogenic HCT in which some patients respond, often with durable remissions. Therefore, the concept that the graft can exert antitumor effects is well established in the field and is a mainstay of the mechanism of how allogenic HCT can potentially cure patients with complex, often refractory, hematological malignancies. Despite the acceptance of the concept of GvL, there is no consensus on the cells responsible for this effect other than T cells are clearly involved or of the target structures recognized on the tumor cells.

Stem cell transplantation (SCT), sometimes referred to as bone marrow transplant (BMT), is a procedure in which a patient receives healthy stem cells (autologous or allogeneic)) to replace damaged stem cells. Before SCT, the patient receives high doses of chemotherapy, and sometimes radiation therapy, to prepare the body for transplantation. This is called “conditioning treatment.” After the stem cells are infused, they will travel in the bloodstream to the bone marrow. These stem cells begin to divide and make new blood cells in the bone marrow, a process called “engraftment.” Engraftment usually happens within the first 30 days after transplantation, but sometimes it can take longer. The doctor will check the patient's blood counts every day to see if the patient's bone marrow has begun producing new blood cells. As engraftment occurs, the numbers of white blood cells, red blood cells and platelets begin to increase in the patient.

### Infectious Disease Vaccines/Adjuvant

The compositions and methods described herein can be used as an adjuvant for infectious disease vaccines to reduce or prevent infections in a human. The majority of vaccine candidates in clinical development are highly purified antigenic proteins and peptides. With their poor immunogenicity alone (primarily related to the removal of danger signals and their small size), immunological adjuvants may be needed to enhance and/or direct immune responses.

Infectious (or communicable) diseases are caused by infectious agents passed from one person (or animal) to another, and these play a huge impact on the health of humans worldwide. These infections are caused by bacteria, parasites, viruses, or fungi (or their toxic by-products), where transmission occurs directly, indirectly or by means of a vector (e.g., mosquitoes).

Vaccines can comprise an antigenic preparation that can include traditional vaccines comprised of whole, killed (or live-attenuated) organisms, or antigenic subunit vaccines using small specific parts of an infectious antigen, which show immense commercial potential with minimal side effects. The antigenic preparation can comprise an antigenic protein, nucleic acid, other subunit of the infectious agent that elicits an immune response. In a nonlimiting example, an antigenic preparation can include the protein, gene, or RNA preparation for hemagglutinin (HA), neuraminidase (NA), or a combination thereof of influenza virus, S protein of Coronavirus, G protein of Respiratory Syncytial Virus (RSV), PspA of Streptococcus pneumonia, HSP65 from Mycobacterium tuberculosis, or IRP1-3 of Bordetella pertussis.

In embodiments, the antigenic preparation can comprise an antigen-encoding RNA preparation. For example, the antigen-encoding RNA preparation can comprise a piece of mRNA encoding an antigenic protein from an infectious disease agent. In embodiments, the mRNA can be encapsulated in a lipid and internalized by human cells. The cells then produce the viral protein, which is recognized by the immune system.

### Compounds for Use in the Methods

In all of the compounds disclosed, the compound may be as depicted, or as a pharmaceutically acceptable salt or ester thereof.

Thus, “alkyl” group refers to a linear, cyclic or branched saturated hydrocarbon, for example a hydrocarbon having from 1 to 10 carbon atoms, in which the atom directly attached to the central structure is a carbon atom. Such an alkyl group may include substituents other than hydrogen, for example an oxygen-containing group including without limitation hydroxyl and alkoxy; a halogen group; a nitrogen-containing group including without limitation amino, amide and alkylamino; an aryl group; a sulfur-containing group including without limitation thioalkyl; and/or a non-aromatic cyclic group including heterocycles and carbocycles. Carbon atoms in these substituents may increase the total number of carbon atoms in the alkyl group to above 10 without departing from the invention. All references to alkyl groups in the specification and claims hereof encompass both substituted and unsubstituted alkyl groups unless the context is clearly to the contrary.

“Alkenyl” group refers to a linear, cyclic or branched hydrocarbon, for example a hydrocarbon having from 1 to 10 carbon atoms, and at least one double bond, in which the atom directly attached to the central structure is a carbon atom. The alkenyl group may include any of the substituents mentioned above for an alkyl group. All references to alkenyl groups in the specification and claims hereof encompass both substituted and unsubstituted alkenyl groups unless the context is clearly to the contrary.

“Alkynyl” group refers to a linear, cyclic or branched hydrocarbon, for example a hydrocarbon having from 1 to 10 carbon atoms, and at least one triple bond, in which the atom directly attached to the central structure is a carbon atom. The alkynyl group may include any of the substituents mentioned above for an alkyl group. All references to alkynyl groups in the specification and claims hereof encompass both substituted and unsubstituted alkynyl groups unless the context is clearly to the contrary.

“Aryl” group refers to any group derived from a simple aromatic ring. Aryl group includes heteroaryl. Aryl groups may be substituted or unsubstituted. When X2, X1 and R is identified as an aryl group, an atom of the aryl ring is bound directly to an atom of the central structure. An aryloxy substituent is an aryl group connected to the central structure through an oxygen atom. The aryl group may include any of the substituents mentioned above for an alkyl group, and in addition an aryl group may include an alkyl, alkenyl or alkynyl group. All references to aryl groups in the specification and claims hereof encompass both substituted and unsubstituted aryl groups unless the context is clearly to the contrary.

“Amino” group refers to any group which consists of a nitrogen attached by single bonds to carbon or hydrogen atoms. In certain instances, the nitrogen of the amino group is directly bound to the central structure. In other instances, an amino group may be a substituent on or within a group, with the nitrogen of the amino group being attached to the central structure through one or more intervening atoms. Examples of amino groups include NH2, alkylamino, alkenylamino groups and N-containing non-aromatic heterocyclic moiety (i.e., cyclic amines). Amino groups may be substituted or unsubstituted. All references to amino groups in the specification and claims hereof encompass substituted and unsubstituted amino groups unless the context is clearly to the contrary.

“Halogen” group refers to fluorine, chlorine, bromine or iodine.

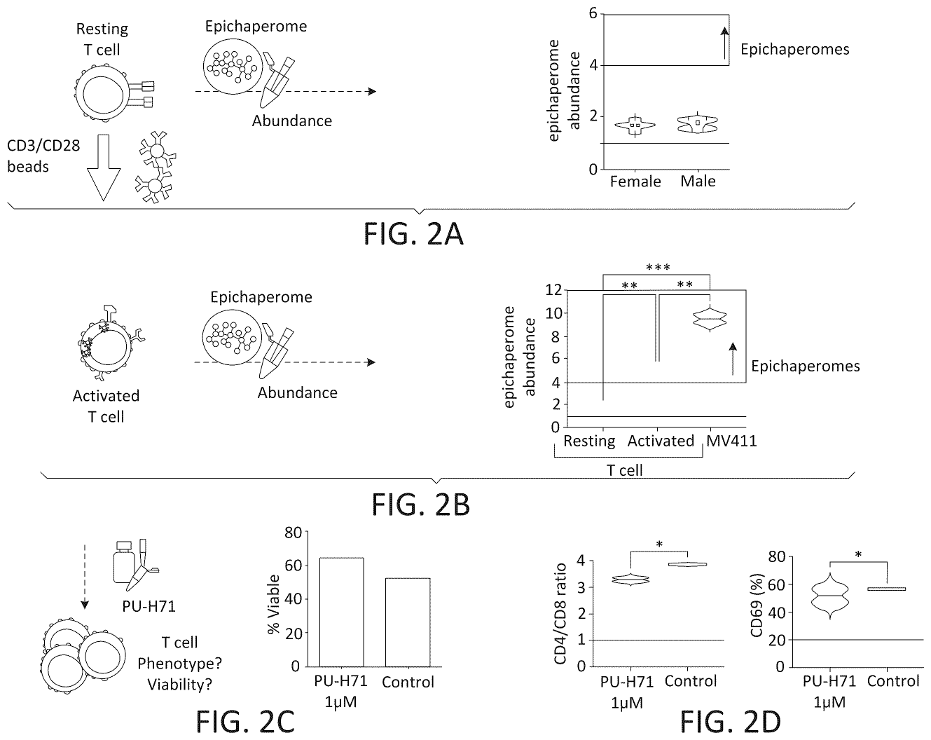

“Heterocyclic” group refers to a moiety containing at least one atom of carbon, and at least one atom of an element other than carbon, such as sulfur, oxygen or nitrogen within a ring structure. These heterocyclic groups may be either aromatic rings or saturated and unsaturated non-aromatic rings. Some examples are given in FIG. 2 of WO2011044394. Heterocylic groups may be substituted or unsubstituted. All references to heterocyclic groups in the specification and claims encompass substituted and unsubstituted heterocyclic groups unless the context is clearly to the contrary.

In the compounds of the disclosure, all of the atoms have sufficient hydrogen or nonhydrogen substituents to satisfy valence, or the compound includes a pharmaceutically acceptable counterion, for example in the case of a quaternary amine.

In the structures set forth below examples are provided in which all of Z₁, Z₂ and Z₃ are nitrogen. These examples are intended as exemplary and are not intended to exclude options in which one or more of Z₁, Z₂ and Z₃ is carbon. In particular, corresponding compositions in which Z₂ or Z₃ is carbon are considered to be within the scope of this disclosure.

Many of these compounds are based on a scaffold of the type disclosed by Chiosis et al. in PCT Publication No. WO02/36075, with variations in substituents. In some cases, the compositions can be described by one of the following two general formulas:

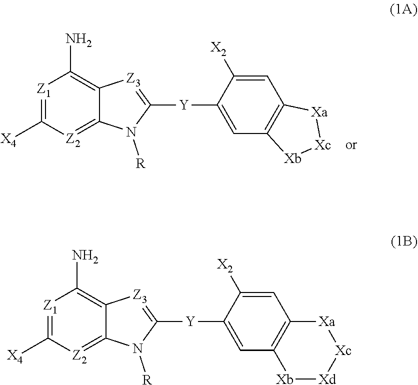

wherein Z₁, Z₂, Z₃ are selected from C and N in which numerous options are disclosed for each variable substituent, resulting in an astronomical number of combinations and permutations. In other cases, the compositions can be described by a structural formula in which Xa, Xb, Xc and Xd are not connected to one another but are simply substituents on the benzene ring. These structures have the general formula:

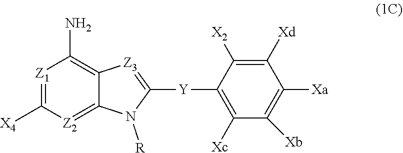

wherein Z₁, Z₂, Z₃ are selected from C and N.
   A. Structures of Formula 1A in which Xa or Xb is O

In some embodiments, the compounds have general formula 1A, in which one of Xa or Xb is O, and Xc and the other of Xa and Xb is CH₂. Thus, the compounds of this embodiment may be

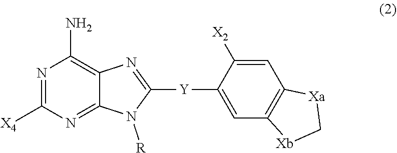

represented by the general formula
wherein:
   one of Xa and Xb is O and the other is —CH:
   Y is —CH₂— or —S—,
   X₄ is hydrogen or halogen; and
   X₂ and R are in combinations as discussed in WO2011044394 pages 8-19.
      B. Structures of Formula 1A in which Xa or Xb is S

In embodiments, the compounds have general formula IA, in which one of Xa or Xb is S, and Xc and the other of Xa and Xb is CH₂. Thus, the compounds of this embodiment may be

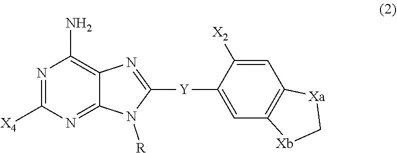

represented by the general formula
wherein:
   one of Xa and Xb is S and the other is —CH₂—;
   Y is —CH₂— or —S—,
      X₄ is hydrogen or halogen; and
   X₂ and R are as discussed in WO2011044394 pages 19-24.
      C. Structures of Formula 1A in which Xa, Xb and Xc are all Carbon

In embodiments, the compounds have general formula 1A; in which Xa, Xb and Xc are all carbon, connected by two single or one single bond and one double bond, and wherein
Y is —CH2- or —S—;
X₄ is hydrogen or halogen; and
X₂ and R are in combinations as discussed in WO2011044394 pages 24-31.
   D. Structures of Formula 1A in which Xa or Xb is N

In embodiments, the compounds have general formula 1A, in which one of Xa or Xb is N, and Xc and the other of Xa and Xb are CH₂. The N may be unsubstituted (i.e. NH) or substituted, for example with methyl, ethyl, acetyl. Thus, the compounds of this embodiment may be represented by the general formula

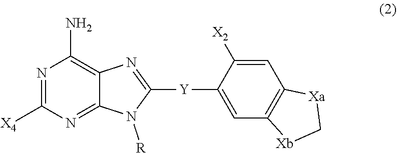

wherein:
   one of Xa and Xb is N bonded to H or a substituent, and the other is —CH₂—;
   Y is —CH₂— or —S—,
   X₄ is hydrogen or halogen; and
   X₂ and Rare as discussed in WO2011044394 pages 31-35.
      E. Structures of Formula 1A in which Xa or Xb is Carbonyl or Thiocarbonyl

In embodiments, the compounds have general formula 1A, in which one of Xa or Xb is carbonyl (C═O) or thiocarbonyl (C═S), and Xc and the other of Xa and Xb is CH₂. Thus, the compounds of this embodiment may be represented by the general formula

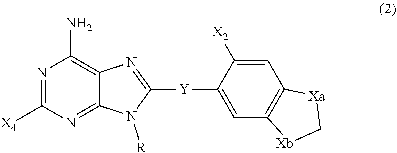

wherein:
   one of Xa and Xb is C═O or C═S, and the other is —CH₂—;
   Y is —CH₂— or —S—,
   X₄ is hydrogen or halogen; and
   X₂ and R are as discussed in WO2011044394 pages 35-42.
      F. Structures of Formula 1A in which Xa, Xb and Xc Include at Least One Noncarbon Atom and -Xa-Xc-Xb- Includes a Double Bond.

In embodiments, the compounds have general formula 1A, with Xa, Xc, Xb and the bonds between them selected from a combination in the following table:

| Xa | Bond | Xc | bond | Xb |
|---|---|---|---|---|
| C | double | C | single | O |
| C | double | C | single | N |
| C | double | C | single | S |
| O | single | C | double | C |
| N | single | C | double | C |
| S | single | C | double | C |
| N | double | C | single | O |
| N | double | C | single | S |
| N | single | C | double | N |
| O | single | C | double | N |
| S | single | C | double | N |
| N | double | N | single | O |
| N | double | N | single | S |
| N | double | N | single | C |
| O | single | N | double | N |
| N | single | N | double | N |
| S | single | N | double | N |
| C | single | N | double | N |

Y is —CH₂— or —S—,
X₄ is hydrogen or halogen; and
X₂ and R are as discussed in WO2011044394 pages 43-48.
   G. Structures of Formula 1A in which Xa and Xb are Both 0

In embodiments, the compounds have general formula 1A, in which Xa and Xb are O and Xc is CH₂. Thus, the compounds of this embodiment may be represented by the general formula

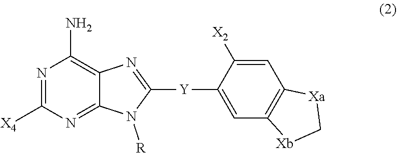

wherein:
   Xa and Xb are O,
   Y is —CH₂— or —S—,
   X₄ is hydrogen or halogen; and
   X₂ and R are in combinations as discussed in WO2011044394 pages 48-59.
      H. Structures of Formula 1B in which Xa and Xb are O

In embodiments, the compounds have general formula 1B, in which both Xa and Xb are O, and Xc and Xd are CH₂. Thus, compounds of this embodiment are represented by the formula:

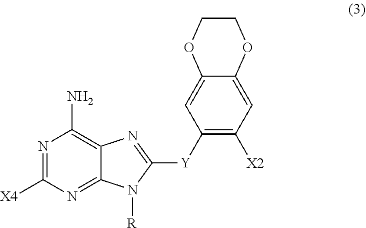

wherein
   Y is —CH₂—, —O— or —S—;
   X₄ is hydrogen or halogen; and
   X₂ and R are as discussed in WO2011044394 pages 59-66.
      I. Structures of Formula 1B in which Xa and/or Xb is a Heteroatom, but not Both O

In accordance with a ninth embodiment of the invention, the compounds have general formula 1B, in which one or both of Xa or Xb is a heteroatom such as O, N or S, with the proviso that both of Xa and Xb are not O (see Section H), and as discussed in discussed in WO2011044394 pages 66-71. Specific examples of compounds within this group are shown in Table 6A of WO2011044394.

J. Structures of Formula 1B in which Xa, Xe, Xd and Xb are all Carbon

In accordance with a tenth embodiment of the invention, the compounds have general formula 1 B, in which Xa, Xe, Xd and Xb are all carbon connected by single or double bonds. In these embodiments,⋅R may be any of the groups disclosed as a substituent at the 9-position nitrogen herein or in the various patents and applications cited in WO2011044394, and as discussed in WO2011044394 pages 71-72. (See Table 8 of WO2011044394)

### K. Structures of Formula 1C

In some embodiments of the present invention having the general formula 1C, the compounds of the invention can be represented by the general formula:

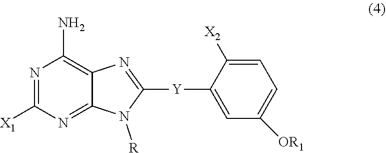

wherein
   R₁ is alkyl, for example methyl or ethyl,
   Y is S or CH₂,
   X₄ is H or halogen,
   X₂ is saturated or unsaturated non-aromatic carbocycle or heterocycle, aryl, alkylamino, dialkylamino, alkynyl or part of a ring formed by R; and R is hydrogen, alkyl, alkenyl, or alkynyl, linear, branched or cyclic, optionally including heteroatoms such as N, Sor O, optionally connected to the 2′-position to form an 8 to 10 member ring.

Various embodiments are discussed in WO2011044394 pages 73-75.

L. Structures in which X2 is Alkynyl

In embodiments, compounds are provided in accordance with any of formulas (1A), (1B) or (1C) in which X₂ is alkynyl. Various embodiments are discussed in WO2011044394 page 75.

M. Structures in which X2 is Furan, Thiophene, Pyrazole, Oxazole or Thiazole.

In embodiments, compounds are provided in accordance with any of formulas (IA), (1B) or (IC) in which X₂ is a furan, thiophene, 3-pyrazole, oxazole or thiazole. Various embodiments are discussed in WO2011044394 page 75-76.

N. Compounds Incorporated by Reference from WO2012138894, Hereby Incorporated in its Entirety

In embodiments, the compound is a compound as described in embodiments (1)-(7):
(1) A Compound of Formula (IA) or (IB):

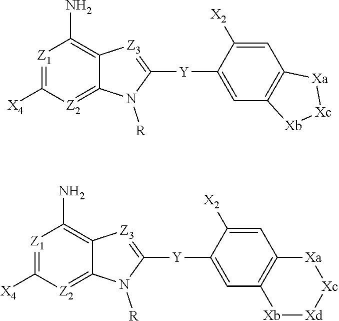

(IA) (IB) or a pharmaceutically acceptable salt thereof, wherein:
(a) each of Z₁ Z2 and Z₃ is independently CH or N;
(b) Y is CH₂, O, or S;
(c) Xa, Xb, Xc and Xd are independently selected from CH, CH₂, O, N, NH, S, carbonyl, fluoromethylene, and difluoromethylene selected so as to satisfy valence, wherein each bond to an X group is either a single bond or a double bond;
(d) X₂ is halogen, aryl, alkynyl, or amino;
(e) X₄ is hydrogen or halogen; and
(f) R is straight-chain- or branched-substituted or unsubstituted alkyl, straight-chain- or branched-substituted or unsubstituted alkenyl, straight-chain- or branched-substituted or unsubstituted alkynyl, or substituted or unsubstituted cycloalkyl wherein the R group is interrupted by —S(O)N(R_{A})—, —NR_{A}S(O)—, —SO₂N(R_{A})—, —NR_{A}SO₂—, —C(O)N(R_{A})—, or —NR_{A}C(O)—, and/or terminated by —S(O)NR_{A}R_{B}, —NR_{A}S(O)R_{B}, —SO₂NR_{A}R_{B}, —NR_{A}SO₂R_{B}. —C(O) NR_{A}R_{B}, or —NR_{A}C(O)R_{B}, wherein each R_{A} and R_{B} is independently selected from hydrogen, C₁-C₆ alkyl, C₂-C₆ alkenyl, C₂-C₆ alkynyl, cycloalkyl, heteroalkyl, heterocycloalkyl, aryl, heteroaryl, alkylaryl, arylalkyl, alkylheteroaryl, heteroarylalkyl, and alkylheteroarylalkyl.
(2) The compound as in the above (1) which is a Compound of Formula (1):

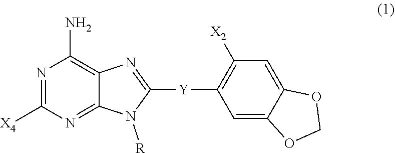

or a pharmaceutically acceptable salt thereof, wherein Y is CH₂ or S.
(3) The compound as in the above (1) which is a Compound of Formula (2):

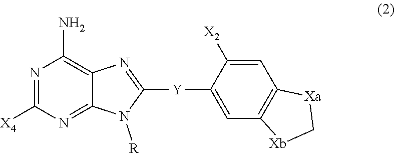

or a pharmaceutically acceptable salt thereof, wherein:
one of Xa and Xb is O and the other is CH₂; and
Y is CH₂ or S.
(4) The compound as in the above (1) which is a Compound of Formula (3):

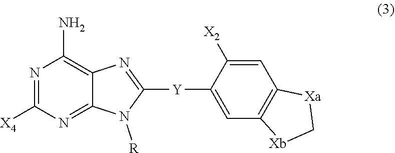

or a pharmaceutically acceptable salt thereof, wherein:
one of Xa and Xb is C(═O) and the other is CH₂; and
Y is CH₂ or S.
(5) The compound as in the above (1) which is a Compound of Formula (4):

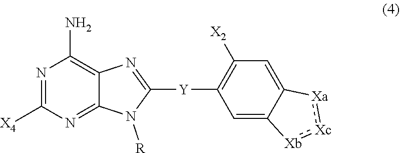

or a pharmaceutically acceptable salt thereof, wherein:
Xa-Xc-Xb is CH₂—CH₂—CH₂, CH═CH—CH₂, or CH₂—CH—CH; and Y is CH₂ or S.
(6) The compound as the above (1) which is a Compound of Formula (5):

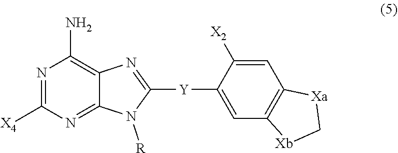

or a pharmaceutically acceptable salt thereof, wherein at least one of Xa and Xb is CHF or CF₂ and the other is CHF, CF₂, or CH₂.
(7) The compound as in the above (1) which is Compound of Formula (6):

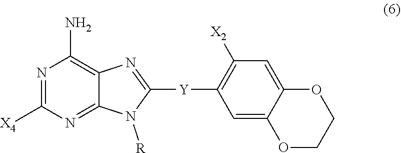

or a pharmaceutically acceptable salt thereof.

Embodiments regarding compounds of Formula (1)-(6) of Section N are described in WO2012138894 pages 6-12 and associated definitions pages 12-20.

O. Compounds Incorporated by Reference from WO2012138896, Hereby Incorporated in its Entirety.

In embodiments, the compound is a compound of Formula (II), and embodiments (1)-(4):
(1) A Compound of Formula (II):

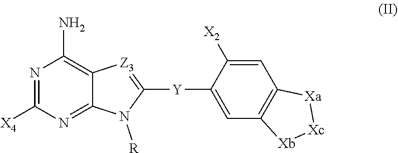

or a pharmaceutically acceptable salt thereof, wherein:
(a) each of Z₁, Z₂, and Z₃ is independently CH or N;
(b) Y is S;
(c) Xa and Xb are O;
(d) X is —CH₂₋;
(e) X₂ is —NR₁R₂, wherein R₁ and R₂ are each independently H, C₁-C₆ alkyl, C₂-C₆ alkenyl, C₂-C₆ alkynyl, cycloalkyl, heteroalkyl, heterocycloalkyl, aryl, heteroaryl, alkylaryl, arylalkyi, alkylheteroaryl, heteroarylalkyl, or alkylheteroarylalkyl, or a protonated form thereof;
(f) X₄ is hydrogen or halogen; and
(g) R is —R₁₀—NH—R₁₁ wherein R₁₀ is ethylene or propylene, and R₁₁ is a branched alkyl.
(2) Compounds of (1) in which Z₁, Z₂ and Z₃ are all N as shown in formula (III)

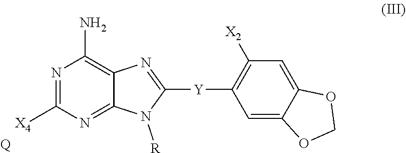

or a pharmaceutically acceptable salt thereof,
(3) Compounds of (1) or (2) or a pharmaceutically acceptable salt thereof in which Rₐ is neopentyl, isopropyl or t-butyl.
(4) Compounds of one of the above (1) to (3) or a pharmaceutically acceptable salt thereof in which X₂ is dimethylamine.

Definitions related to compounds of formula (II) and (III) of section O, can be found in WO2012/138896 pages 7-14.

In some embodiments, the compound is selected from:

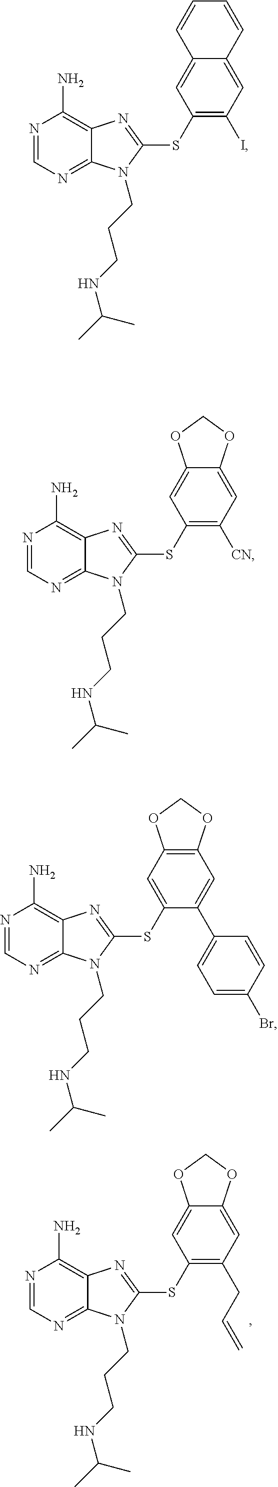 [SMILES: CC(C)NCCCn1c(Sc2cc3ccccc3cc2I)nc2c(N)ncnc21 ; CC(C)NCCCn1c(Sc2cc3c(cc2C#N)OCO3)nc2c(N)ncnc21 ; CC(C)NCCCn1c(Sc2cc3c(cc2-c2ccc(Br)cc2)OCO3)nc2c(N)ncnc21 ; C=CCc1cc2c(cc1Sc1nc3c(N)ncnc3n1CCCNC(C)C)OCO2]

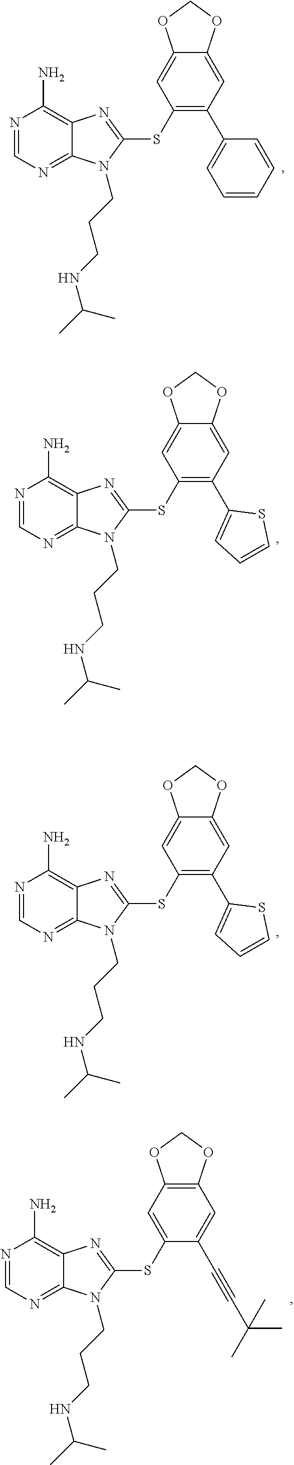 [SMILES: CC(C)NCCCn1c(Sc2cc3c(cc2-c2ccccc2)OCO3)nc2c(N)ncnc21 ; CC(C)NCCCn1c(Sc2cc3c(cc2-c2cccs2)OCO3)nc2c(N)ncnc21 ; CC(C)NCCCn1c(Sc2cc3c(cc2-c2cccs2)OCO3)nc2c(N)ncnc21 ; CC(C)NCCCn1c(Sc2cc3c(cc2C#CC(C)(C)C)OCO3)nc2c(N)ncnc21]

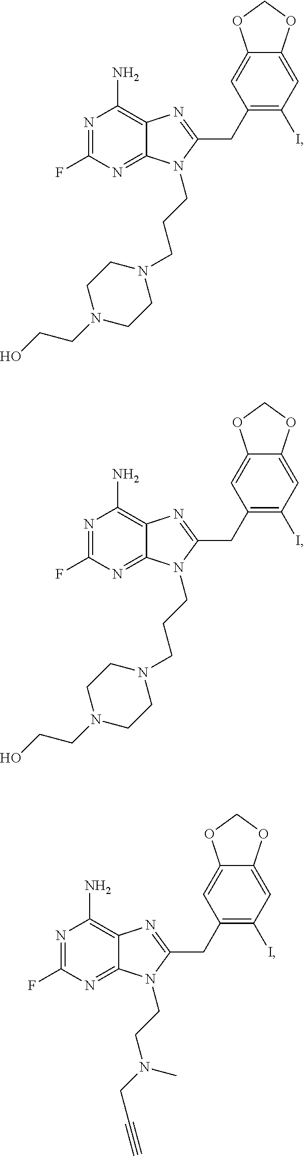 [SMILES: Nc1nc(F)nc2c1nc(Cc1cc3c(cc1I)OCO3)n2CCCN1CCN(CCO)CC1 ; Nc1nc(F)nc2c1nc(Cc1cc3c(cc1I)OCO3)n2CCCN1CCN(CCO)CC1 ; C#CCN(C)CCn1c(Cc2cc3c(cc2I)OCO3)nc2c(N)nc(F)nc21]

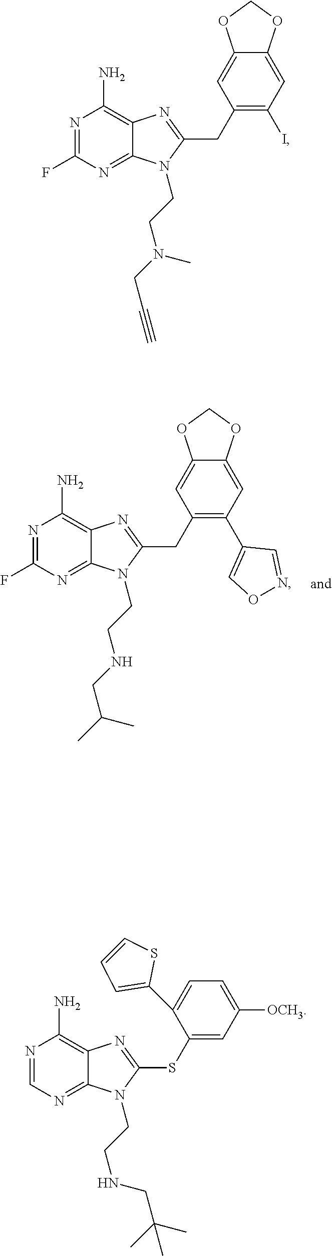 [SMILES: C#CCN(C)CCn1c(Cc2cc3c(cc2I)OCO3)nc2c(N)nc(F)nc21 ; CC(C)CNCCn1c(Cc2cc3c(cc2-c2cnoc2)OCO3)nc2c(N)nc(F)nc21 ; COc1ccc(-c2cccs2)c(Sc2nc3c(N)ncnc3n2CCNCC(C)(C)C)c1]

In some embodiments, the compound is selected from:

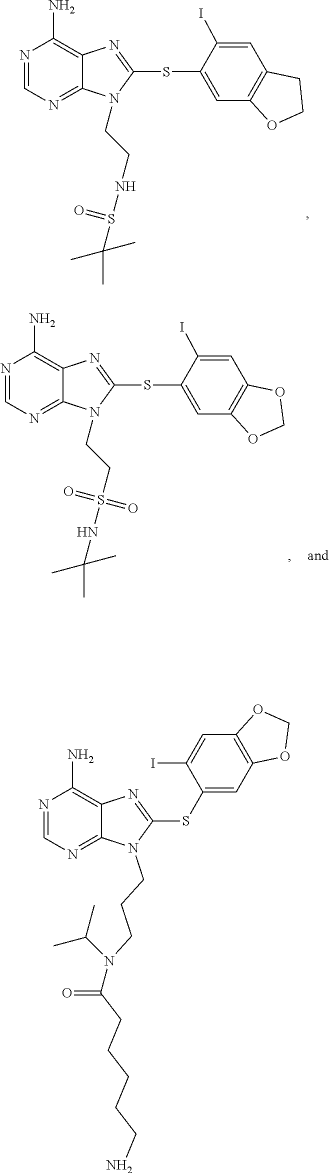 [SMILES: CC(C)(C)S(=O)NCCn1c(Sc2cc3c(cc2I)CCO3)nc2c(N)ncnc21 ; CC(C)(C)NS(=O)(=O)CCn1c(Sc2cc3c(cc2I)OCO3)nc2c(N)ncnc21 ; CC(C)N(CCCn1c(Sc2cc3c(cc2I)OCO3)nc2c(N)ncnc21)C(=O)CCCCCN]

In some embodiments, the compound is selected from:

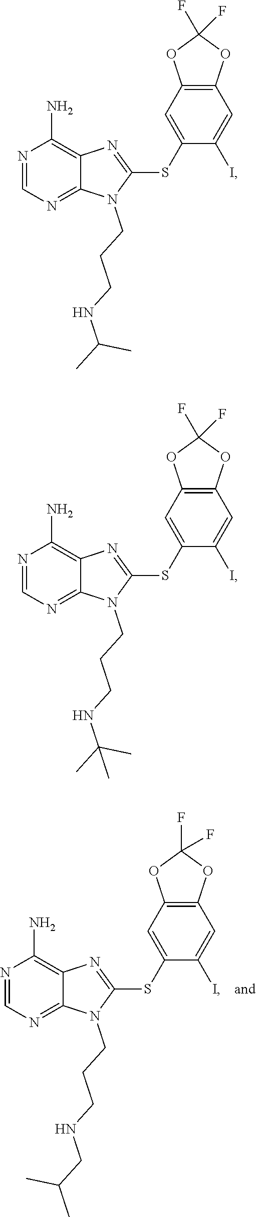 [SMILES: CC(C)NCCCn1c(Sc2cc3c(cc2I)OC(F)(F)O3)nc2c(N)ncnc21 ; CC(C)(C)NCCCn1c(Sc2cc3c(cc2I)OC(F)(F)O3)nc2c(N)ncnc21 ; CC(C)CNCCCn1c(Sc2cc3c(cc2I)OC(F)(F)O3)nc2c(N)ncnc21]

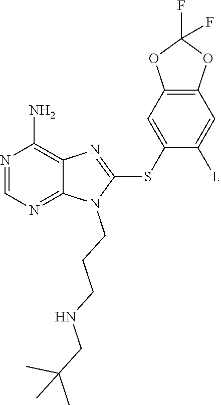 [SMILES: CC(C)(C)CNCCCn1c(Sc2cc3c(cc2I)OC(F)(F)O3)nc2c(N)ncnc21]

In some embodiments, the compound is selected from:

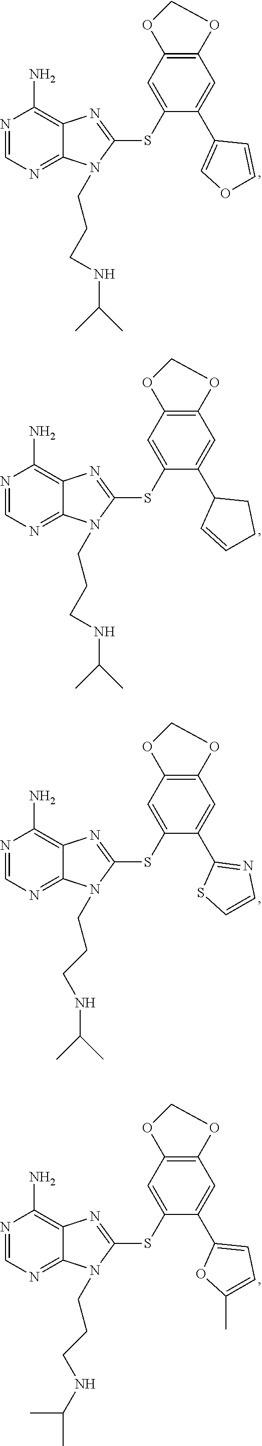 [SMILES: CC(C)NCCCn1c(Sc2cc3c(cc2-c2ccoc2)OCO3)nc2c(N)ncnc21 ; CC(C)NCCCn1c(Sc2cc3c(cc2C2C=CCC2)OCO3)nc2c(N)ncnc21 ; CC(C)NCCCn1c(Sc2cc3c(cc2-c2nccs2)OCO3)nc2c(N)ncnc21 ; Cc1ccc(-c2cc3c(cc2Sc2nc4c(N)ncnc4n2CCCNC(C)C)OCO3)o1]

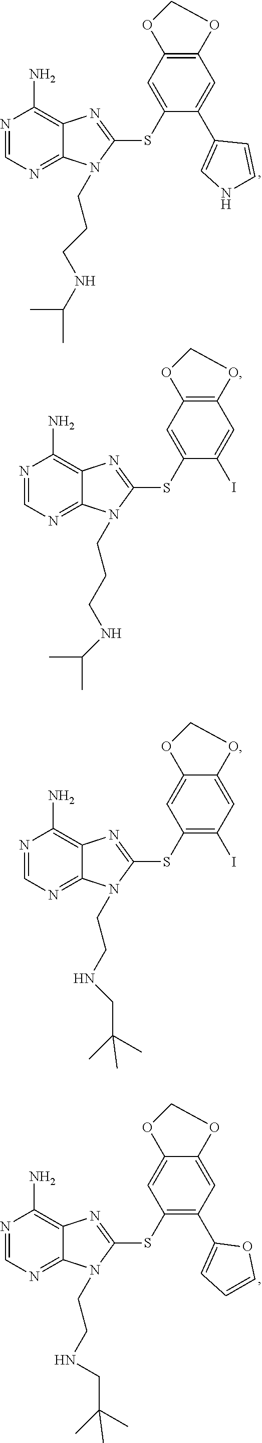 [SMILES: CC(C)NCCCn1c(Sc2cc3c(cc2-c2cc[nH]c2)OCO3)nc2c(N)ncnc21 ; CC(C)NCCCn1c(Sc2cc3c(cc2I)OCO3)nc2c(N)ncnc21 ; CC(C)(C)CNCCn1c(Sc2cc3c(cc2I)OCO3)nc2c(N)ncnc21 ; CC(C)(C)CNCCn1c(Sc2cc3c(cc2-c2ccco2)OCO3)nc2c(N)ncnc21]

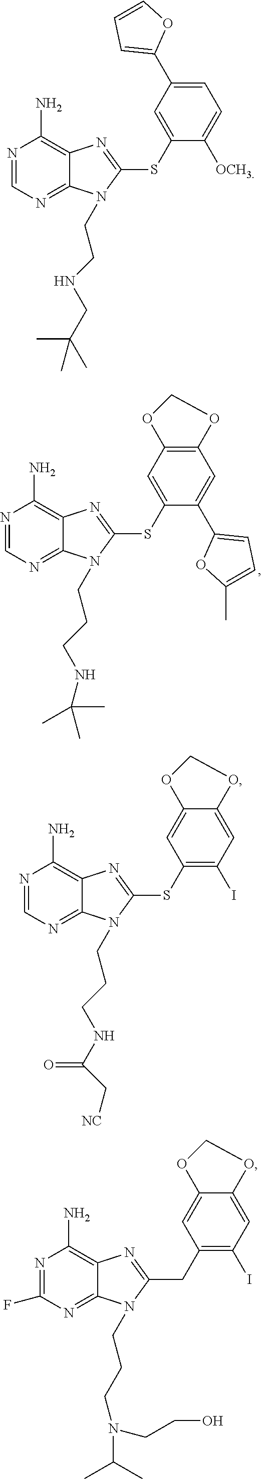 [SMILES: COc1ccc(-c2ccco2)cc1Sc1nc2c(N)ncnc2n1CCNCC(C)(C)C ; Cc1ccc(-c2cc3c(cc2Sc2nc4c(N)ncnc4n2CCCNC(C)(C)C)OCO3)o1 ; N#CCC(=O)NCCCn1c(Sc2cc3c(cc2I)OCO3)nc2c(N)ncnc21 ; CC(C)N(CCO)CCCn1c(Cc2cc3c(cc2I)OCO3)nc2c(N)nc(F)nc21]

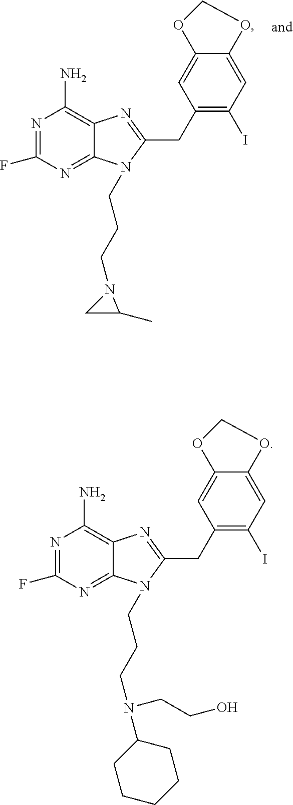 [SMILES: CC1CN1CCCn1c(Cc2cc3c(cc2I)OCO3)nc2c(N)nc(F)nc21 ; Nc1nc(F)nc2c1nc(Cc1cc3c(cc1I)OCO3)n2CCCN(CCO)C1CCCCC1]

In some embodiments, the compound is selected from:

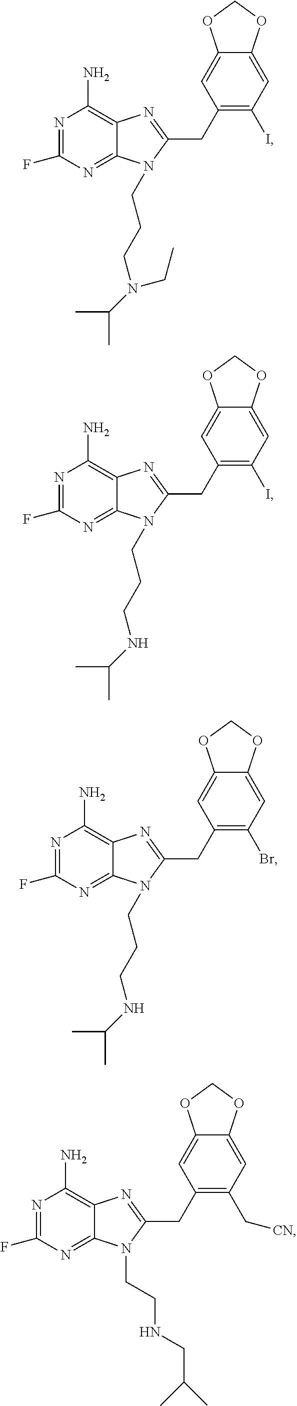 [SMILES: CCN(CCCn1c(Cc2cc3c(cc2I)OCO3)nc2c(N)nc(F)nc21)C(C)C ; CC(C)NCCCn1c(Cc2cc3c(cc2I)OCO3)nc2c(N)nc(F)nc21 ; CC(C)NCCCn1c(Cc2cc3c(cc2Br)OCO3)nc2c(N)nc(F)nc21 ; CC(C)CNCCn1c(Cc2cc3c(cc2CC#N)OCO3)nc2c(N)nc(F)nc21]

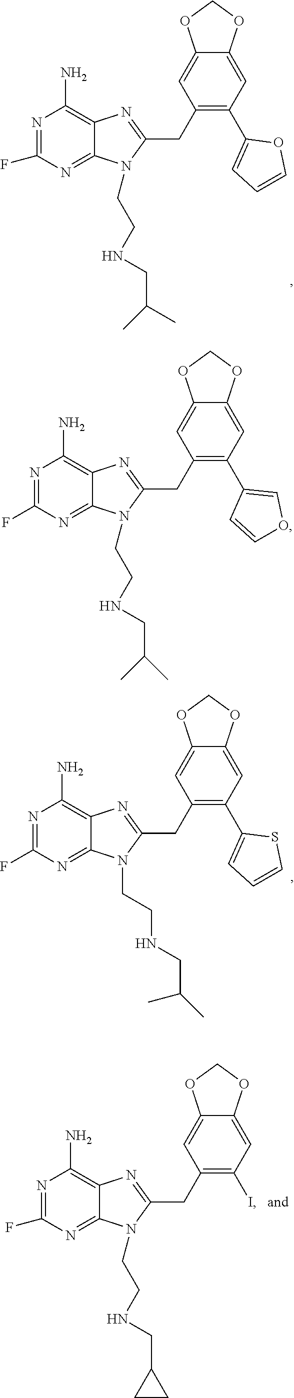

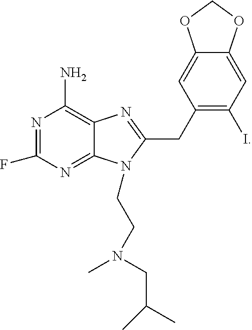 [SMILES: CC(C)CN(C)CCn1c(Cc2cc3c(cc2I)OCO3)nc2c(N)nc(F)nc21]

In some embodiments, the compound is selected from:

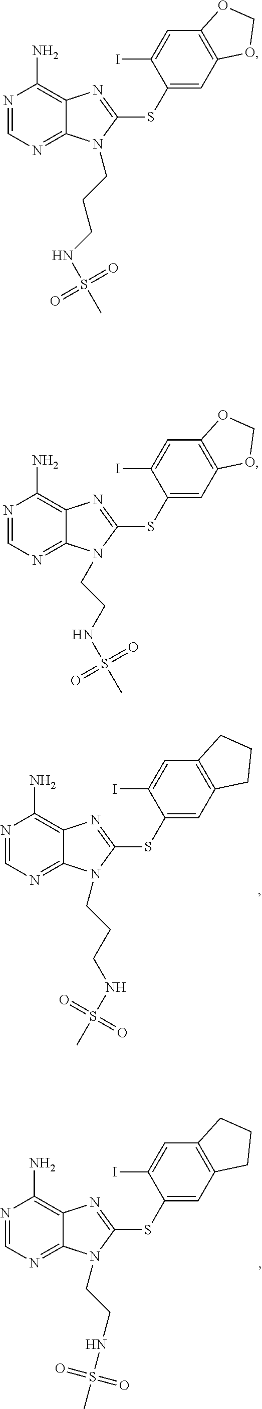 [SMILES: CS(=O)(=O)NCCCn1c(Sc2cc3c(cc2I)OCO3)nc2c(N)ncnc21 ; CS(=O)(=O)NCCn1c(Sc2cc3c(cc2I)OCO3)nc2c(N)ncnc21 ; CS(=O)(=O)NCCCn1c(Sc2cc3c(cc2I)CCC3)nc2c(N)ncnc21 ; CS(=O)(=O)NCCn1c(Sc2cc3c(cc2I)CCC3)nc2c(N)ncnc21]

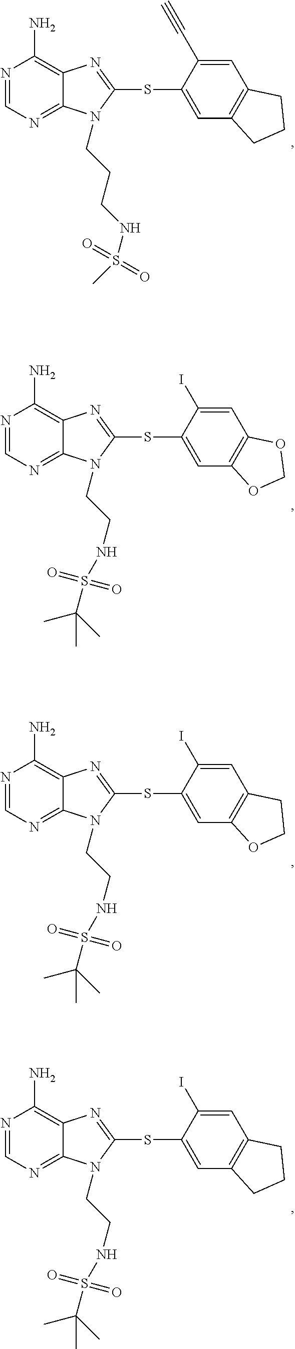 [SMILES: C#Cc1cc2c(cc1Sc1nc3c(N)ncnc3n1CCCNS(C)(=O)=O)CCC2 ; CC(C)(C)S(=O)(=O)NCCn1c(Sc2cc3c(cc2I)OCO3)nc2c(N)ncnc21 ; CC(C)(C)S(=O)(=O)NCCn1c(Sc2cc3c(cc2I)CCO3)nc2c(N)ncnc21 ; CC(C)(C)S(=O)(=O)NCCn1c(Sc2cc3c(cc2I)CCC3)nc2c(N)ncnc21]

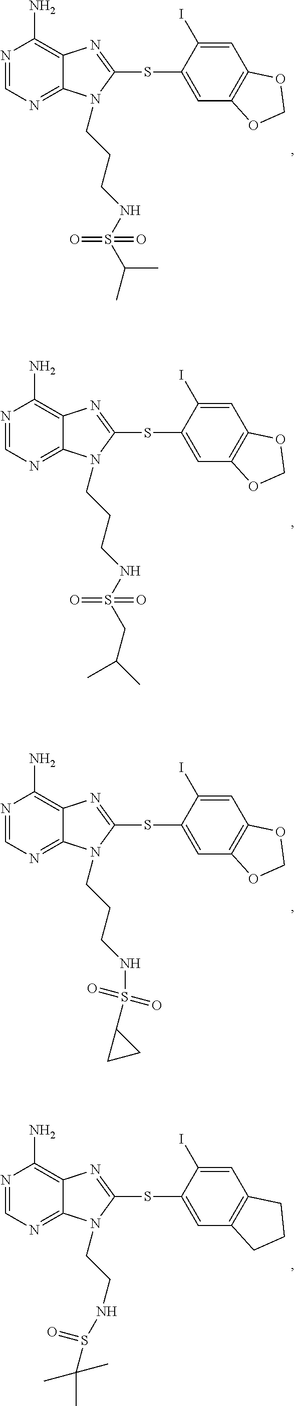 [SMILES: CC(C)S(=O)(=O)NCCCn1c(Sc2cc3c(cc2I)OCO3)nc2c(N)ncnc21 ; CC(C)CS(=O)(=O)NCCCn1c(Sc2cc3c(cc2I)OCO3)nc2c(N)ncnc21 ; Nc1ncnc2c1nc(Sc1cc3c(cc1I)OCO3)n2CCCNS(=O)(=O)C1CC1 ; CC(C)(C)S(=O)NCCn1c(Sc2cc3c(cc2I)CCC3)nc2c(N)ncnc21]

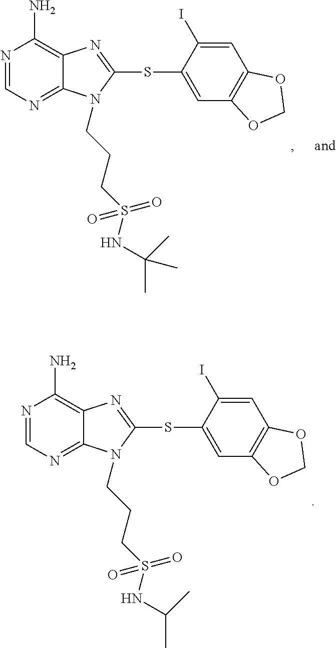 [SMILES: CC(C)(C)NS(=O)(=O)CCCn1c(Sc2cc3c(cc2I)OCO3)nc2c(N)ncnc21 ; CC(C)NS(=O)(=O)CCCn1c(Sc2cc3c(cc2I)OCO3)nc2c(N)ncnc21]

In some embodiments, the compound is selected from:

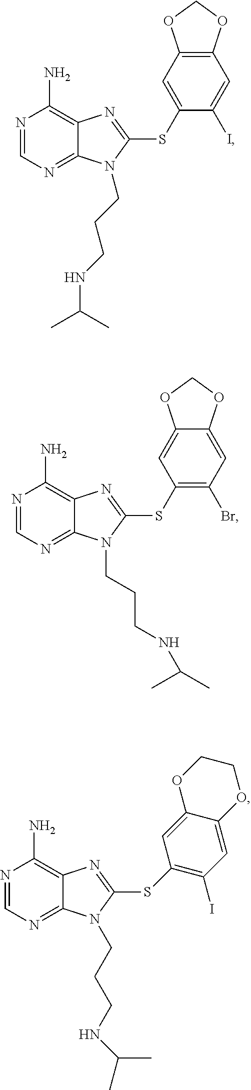 [SMILES: CC(C)NCCCn1c(Sc2cc3c(cc2I)OCO3)nc2c(N)ncnc21 ; CC(C)NCCCn1c(Sc2cc3c(cc2Br)OCO3)nc2c(N)ncnc21 ; CC(C)NCCCn1c(Sc2cc3c(cc2I)OCCO3)nc2c(N)ncnc21]

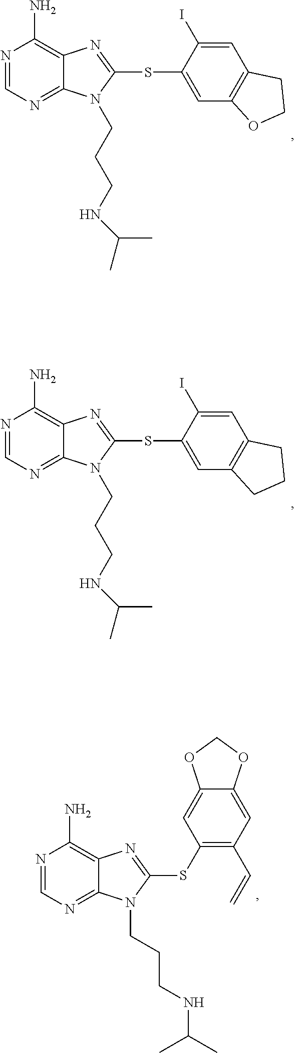 [SMILES: CC(C)NCCCn1c(Sc2cc3c(cc2I)CCO3)nc2c(N)ncnc21 ; CC(C)NCCCn1c(Sc2cc3c(cc2I)CCC3)nc2c(N)ncnc21 ; C=Cc1cc2c(cc1Sc1nc3c(N)ncnc3n1CCCNC(C)C)OCO2]

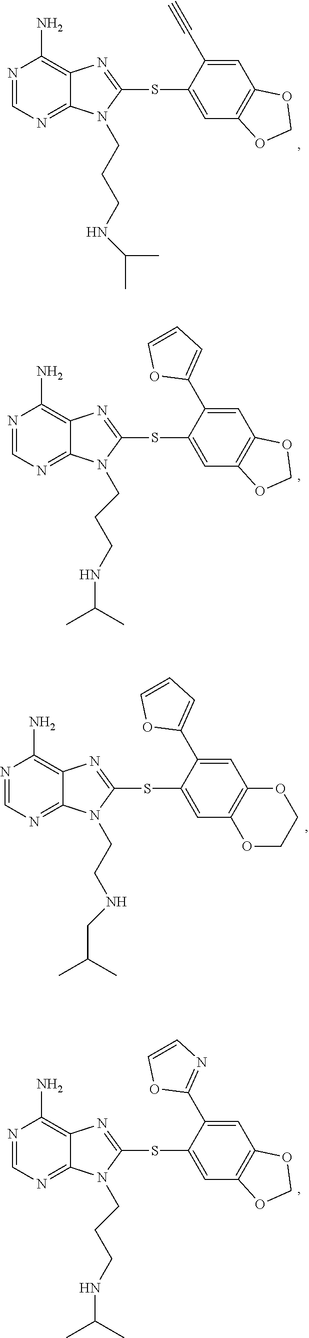 [SMILES: C#Cc1cc2c(cc1Sc1nc3c(N)ncnc3n1CCCNC(C)C)OCO2 ; CC(C)NCCCn1c(Sc2cc3c(cc2-c2ccco2)OCO3)nc2c(N)ncnc21 ; CC(C)CNCCn1c(Sc2cc3c(cc2-c2ccco2)OCCO3)nc2c(N)ncnc21 ; CC(C)NCCCn1c(Sc2cc3c(cc2-c2ncco2)OCO3)nc2c(N)ncnc21]

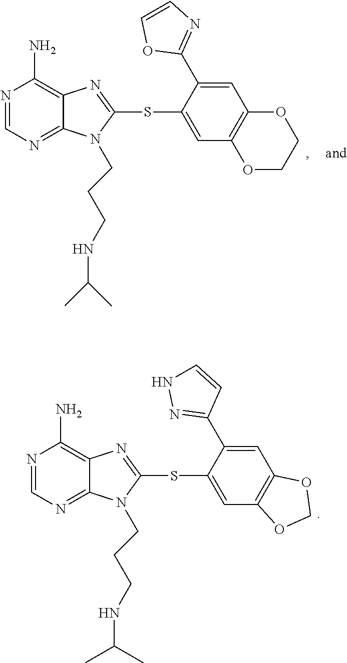 [SMILES: CC(C)NCCCn1c(Sc2cc3c(cc2-c2ncco2)OCCO3)nc2c(N)ncnc21 ; CC(C)NCCCn1c(Sc2cc3c(cc2-c2cc[nH]n2)OCO3)nc2c(N)ncnc21]

In some embodiments, the compound is selected from:

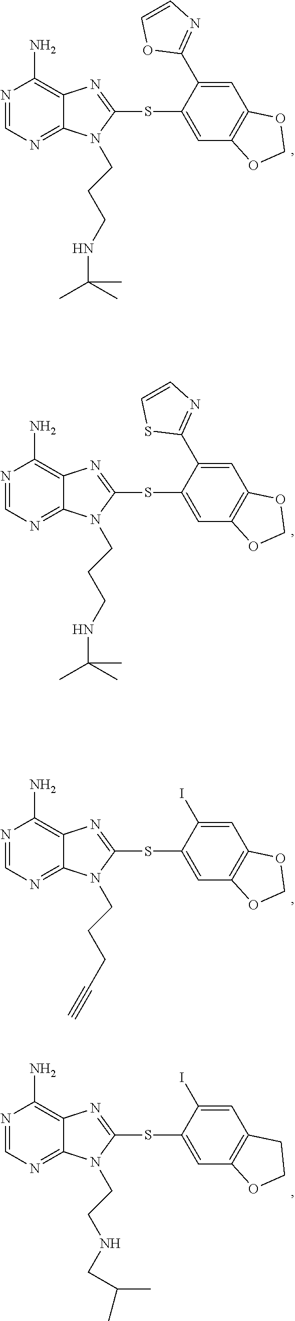 [SMILES: CC(C)(C)NCCCn1c(Sc2cc3c(cc2-c2ncco2)OCO3)nc2c(N)ncnc21 ; CC(C)(C)NCCCn1c(Sc2cc3c(cc2-c2nccs2)OCO3)nc2c(N)ncnc21 ; C#CCCCn1c(Sc2cc3c(cc2I)OCO3)nc2c(N)ncnc21 ; CC(C)CNCCn1c(Sc2cc3c(cc2I)CCO3)nc2c(N)ncnc21]

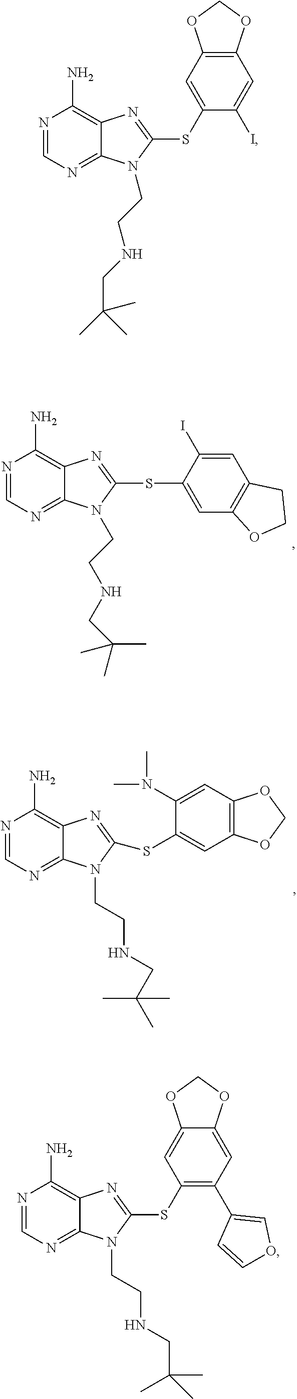 [SMILES: CC(C)(C)CNCCn1c(Sc2cc3c(cc2I)OCO3)nc2c(N)ncnc21 ; CC(C)(C)CNCCn1c(Sc2cc3c(cc2I)CCO3)nc2c(N)ncnc21 ; CN(C)c1cc2c(cc1Sc1nc3c(N)ncnc3n1CCNCC(C)(C)C)OCO2 ; CC(C)(C)CNCCn1c(Sc2cc3c(cc2-c2ccoc2)OCO3)nc2c(N)ncnc21]

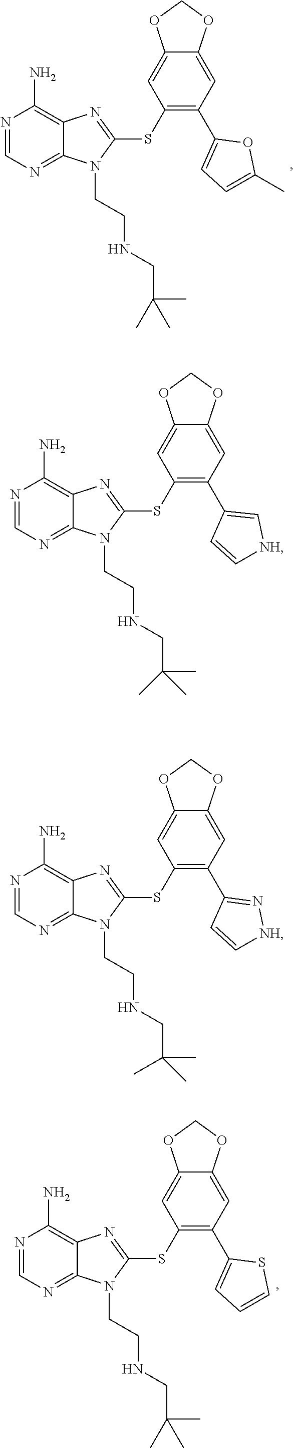

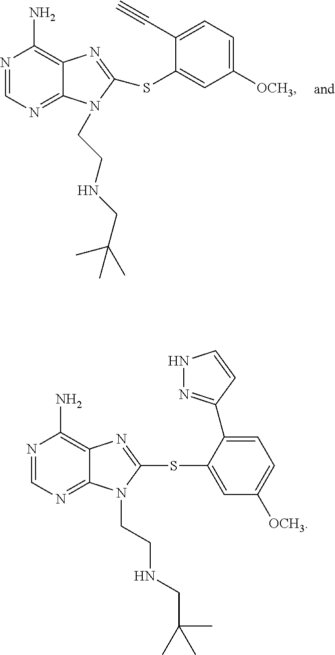 [SMILES: C#Cc1ccc(OC)cc1Sc1nc2c(N)ncnc2n1CCNCC(C)(C)C ; COc1ccc(-c2cc[nH]n2)c(Sc2nc3c(N)ncnc3n2CCNCC(C)(C)C)c1]

In some embodiments, the compound is selected from:

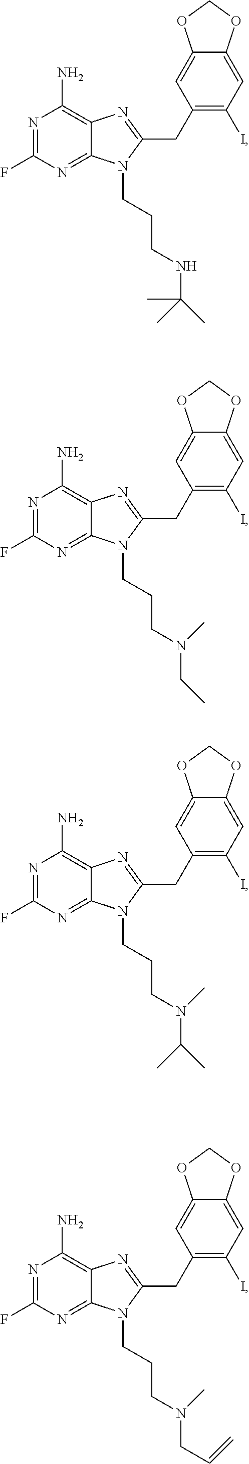

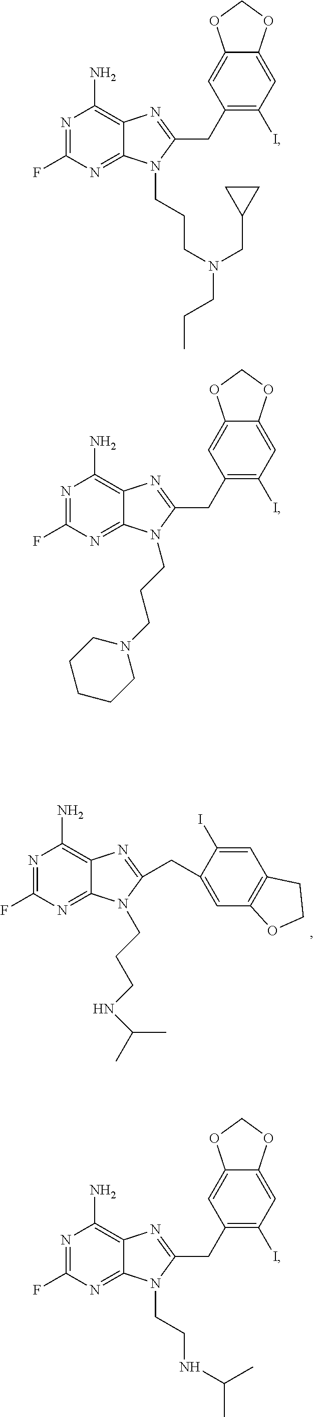 [SMILES: CCCN(CCCn1c(Cc2cc3c(cc2I)OCO3)nc2c(N)nc(F)nc21)CC1CC1 ; Nc1nc(F)nc2c1nc(Cc1cc3c(cc1I)OCO3)n2CCCN1CCCCC1 ; CC(C)NCCCn1c(Cc2cc3c(cc2I)CCO3)nc2c(N)nc(F)nc21 ; CC(C)NCCn1c(Cc2cc3c(cc2I)OCO3)nc2c(N)nc(F)nc21]

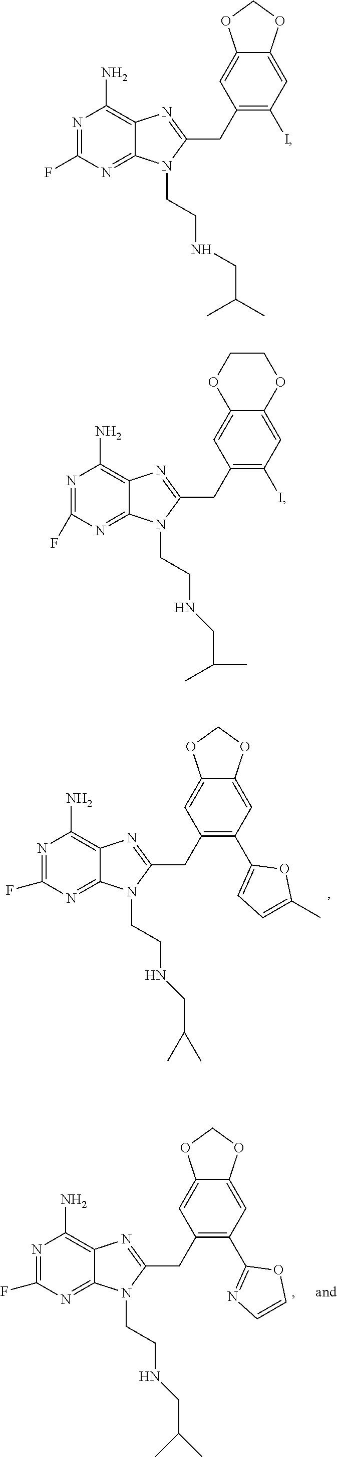

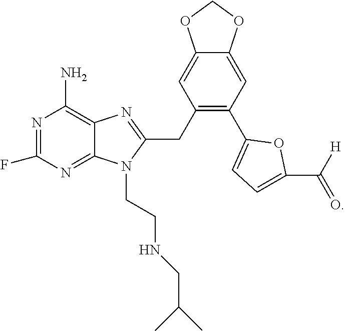 [SMILES: CC(C)CNCCn1c(Cc2cc3c(cc2-c2ccc(C=O)o2)OCO3)nc2c(N)nc(F)nc21]

In some embodiments, the compound is selected from:

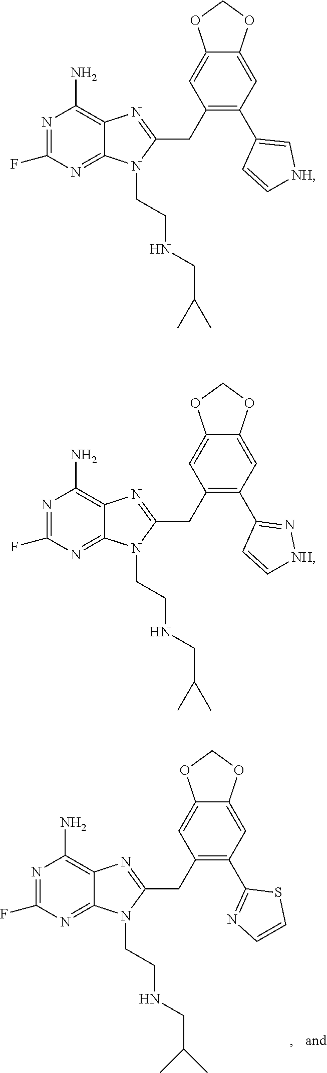

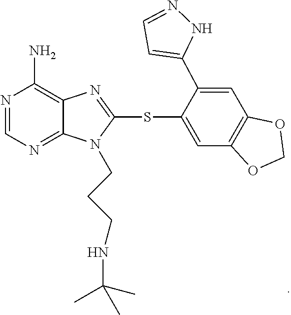 [SMILES: CC(C)(C)NCCCn1c(Sc2cc3c(cc2-c2ccn[nH]2)OCO3)nc2c(N)ncnc21]

In some embodiments, the compound is

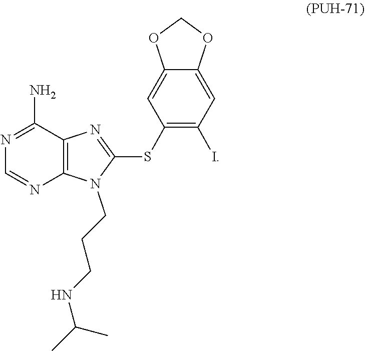 [SMILES: CC(C)NCCCn1c(Sc2cc3c(cc2I)OCO3)nc2c(N)ncnc21]

In one aspect, compounds described herein possess one or more stereocenters and each stereocenter exists independently in either the R or S configuration. The compounds presented herein include all diastereomeric, enantiomeric, and epimeric forms as well as the appropriate mixtures thereof. The compounds and methods provided herein include all cis, trans, syn, anti, entgegen (E), and zusammen (Z) isomers as well as the appropriate mixtures thereof. In certain embodiments, compounds described herein are prepared as their individual stereoisomers by reacting a racemic mixture of the compound with an optically active resolving agent to form a pair of diastereoisomeric compounds/salts, separating the diastereomers and recovering the optically pure enantiomers. In some embodiments, resolution of enantiomers is carried out using covalent diastereomeric derivatives of the compounds described herein. In another embodiment, diastereomers are separated by separation/resolution techniques based upon differences in solubility. In other embodiments, separation of steroisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley and Sons, Inc., 1981. In some embodiments, stereoisomers are obtained by stereoselective synthesis.

The methods and compositions described herein include the use of amorphous forms as well as crystalline forms (also known as polymorphs). In one aspect, compounds described herein are in the form of pharmaceutically acceptable salts. As well, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

In some embodiments, compounds described herein are prepared as prodrugs. A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. In some embodiments, the design of a prodrug increases the effective water solubility. An example, without limitation, of a prodrug is a compound described herein, which is administered as an ester (the “prodrug”) but then is metabolically hydrolyzed to provide the active entity. In some embodiments, the active entity is a phenolic compound as described herein. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

Prodrugs of the compounds described herein include, but are not limited to, esters, ethers, carbonates, thiocarbonates, N-acyl derivatives, N-acyloxyalkyl derivatives, quaternary derivatives of tertiary amines, N-Mannich bases, Schiff bases, amino acid conjugates, phosphate esters, and sulfonate esters. See for example Design of Prodrugs, Bundgaard, A. Ed., Elseview, 1985 and Method in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, each of which is incorporated herein by reference. In some embodiments, a hydroxyl group in the compounds disclosed herein is used to form a prodrug, wherein the hydroxyl group is incorporated into an acyloxyalkyl ester, alkoxycarbonyloxyalkyl ester, alkyl ester, aryl ester, phosphate ester, sugar ester, ether, and the like. Prodrug forms of the herein described compounds, wherein the prodrug is metabolized in vivo to produce a compound of Formulas as set forth herein are included within the scope of the claims. In some cases, some of the herein-described compounds may be a prodrug for another derivative or active compound.

In additional or further embodiments, the compounds described herein are metabolized upon administration to an organism in need to produce a metabolite that is then used to produce a desired effect, including a desired therapeutic effect.

The term “pharmaceutically acceptable salt” refers to a formulation of a compound that does not cause significant irritation to an organism to which it is administered and does not abrogate the biological activity and properties of the compound. In some embodiments, pharmaceutically acceptable salts are obtained by reacting a compound described herein with acids. Pharmaceutically acceptable salts are also obtained by reacting a compound described herein with a base to form a salt. Compounds described herein may be formed as, and/or used as, pharmaceutically acceptable salts. The type of pharmaceutical acceptable salts, include, but are not limited to: (1) acid addition salts, formed by reacting the free base form of the compound with a pharmaceutically acceptable: inorganic acid to form a salt such as, for example, a hydrochloric acid salt, a hydrobromic acid salt, a sulfuric acid salt, a phosphoric acid salt, a metaphosphoric acid salt, and the like; or with an organic acid to form a salt such as, for example, an acetic acid salt, a propionic acid salt, a hexanoic acid salt, a cyclopentanepropionic acid salt, a glycolic acid salt, a pyruvic acid salt, a lactic acid salt, a malonic acid salt, a succinic acid salt, a malic acid salt, a maleic acid salt, a fumaric acid salt, a trifluoroacetic acid salt, a tartaric acid salt, a citric acid salt, a benzoic acid salt, a 3-(4-hydroxybenzoyl)benzoic acid salt, a cinnamic acid salt, a mandelic acid salt, a methanesulfonic acid salt, an ethanesulfonic acid salt, a 1,2-ethanedisulfonic acid salt, a 2-hydroxyethanesulfonic acid salt, a benzenesulfonic acid salt, a toluenesulfonic acid salt, a 2-naphthalenesulfonic acid salt, a 4-methylbicyclo-[2.2.2]oct-2-ene-1carboxylic acid salt, a glucoheptonic acid salt, a 4,4′-methylenebis-(3-hydroxy-2-ene-1-carboxylic acid) salt, a 3-phenylpropionic acid salt, a trimethylacetic acid salt, a tertiary butylacetic acid salt, a lauryl sulfuric acid salt, a gluconic acid salt, a glutamic acid salt, a hydroxynaphthoic acid salt, a salicylic acid salt, a stearic acid salt, a muconic acid salt, a butyric acid salt, a phenylacetic acid salt, a phenylbutyric acid salt, a valproic acid salt, and the like; (2) salts formed when an acidic proton present in the parent compound is replaced by a metal ion, e.g., an alkali metal ion (e.g. a lithium salt, a sodium salt, or a potassium salt), an alkaline earth ion (e.g. a magnesium salt, or a calcium salt), or an aluminum ion (e.g. an aluminum salt). In some cases, compounds described herein may coordinate with an organic base to form a salt, such as, but not limited to, an ethanolamine salt, a diethanolamine salt, a triethanolamine salt, a tromethamine salt, a N-methylglucamine salt, a dicyclohexylamine salt, or a tris(hydroxymethyl)methylamine salt. In other cases, compounds described herein may form salts with amino acids such as, but not limited to, an arginine salt, a lysine salt, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like.

It should be understood that a reference to a pharmaceutically acceptable salt includes the solvent addition forms. Solvates contain either stoichiometric or non-stoichiometric amounts of a solvent and may be formed during the process of crystallization with pharmaceutically acceptable solvents such as water, ethanol, and the like. Hydrates are formed when the solvent is water, or alcoholates are formed when the solvent is alcohol. Solvates of compounds described herein can be conveniently prepared or formed during the processes described herein. In addition, the compounds provided herein can exist in unsolvated as well as solvated forms.

Compounds described herein are synthesized using standard synthetic techniques or using methods known in the art in combination with methods described herein. In additions, solvents, temperatures and other reaction conditions presented herein may vary.

The methods described herein include the use of pharmaceutical compositions comprising an active ingredient described herein.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants.

Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin. Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules.

Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives.

Transmucosal administration can be accomplished through the use of nasal sprays or suppositories. For transdermal administration, the active compounds are formulated into ointments, salves, gels, or creams as generally known in the art.

Therapeutic compounds that are or include nucleic acids can be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al, Clin. Immunol. Immunopathol., 88 (2), 205-10 (1998). Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

### EXAMPLES

### Example I

Despite recent advances in treating acute myeloid leukemia (AML), a subset of patients remains without viable therapeutic options. Such was the case with a patient diagnosed with a myeloproliferative neoplasm (MPN) who progressed to AML despite undergoing allogeneic stem cell transplantation. The patient's blast and leukemia stem cells exhibited a notable abundance of epichaperomes, aberrant scaffolds that disrupt normal protein assembly in disease states. Notably, a groundbreaking single-patient IND trial utilizing the epichaperome disruptor PU-H71 (termed zelavespib in clinic) induced a complete and sustained remission for over five years. Several remarkable features of zelavespib emerged from the experience in treating this patient: she achieved complete remission; she was treated for >5 yrs, in an ongoing manner, without significant myelosuppression or drug-related extramedullary toxicities; she never required hospitalization; there was no clinically relevant exacerbation of graft vs host disease (GvHD). The patient maintained chimerism of donor-derived T cells, suggesting a possible correlation between the prolonged response and an enhanced donor-derived T cell anti-tumor activity derived from epichaperome disruption by zelavespib, likely facilitating graft vs. leukemia (GvL). This connection was substantiated by preclinical studies, where it was found that T cells assembled epichaperomes upon activation. Furthermore, treatment with PU-H71 skewed T cells to an increase of CD8⁺ cells and improved anti-tumor responses in vitro and in vivo. Based on this premise, it is believed that epichaperome exerts a novel role in T cells and that its disruption can modulate T cell responses and improve the efficacy of T cell immunotherapies.

Provided herein is understanding of the role of the epichaperome in T cell function. The use epichaperome disruptors in the realm of T cell anti-tumor responses, whether in the context of stem cell transplantation (improving GvL) or CAR T cell therapies is transformative. With epichaperome disruptors already in clinic (i.e., zelavespib, icapamespib) and others in late stages of preclinical development, this will provide advancements in the treatment of hematologic malignancies, reshaping the landscape of oncological therapy and patient care.

Acute myeloid leukemia (AML) is a hematologic malignancy with dismal outcomes despite advances in targeted approaches based on gene alterations (1). Most patients will achieve complete remission (CR) after induction therapy using intensive chemotherapy. However, most of those patients are destined to relapse and die of disease (2). Allogeneic hematopoietic stem cell transplant (allo-HSCT) represents a curative treatment for many AML patients as it has been shown that donor cells exert an anti-leukemia activity known as GvL effect (3). Unfortunately, post-transplant relapses are almost always fatal within 6-12 mos: and take place in 30-70% of transplanted AML patients as their graft fails to eliminate the residual disease (4). To date there are no FDA-approved treatments specifically for AML patients who relapse after allo-HSCT, a particularly chemotherapy-resistant subgroup. A significant problem with allo-HSCT is GvHD, which arises from donor derived T cells recognizing the recipient as foreign and causing a range of symptoms, from mild skin rash to life-threatening complications such as damage to the liver and gastrointestinal track (5). As GvL and GvHD go hand in hand, there are increasing efforts to understand how to augment GvL without worsening GvHD in allo-HSCT regimens (6). Consequently, there is an unmet need to improve allo-HSCT success without increasing the risk of GvHD and to identify treatments for patients relapsing after allo-HSCT.

Epichaperomes re a vulnerability of tumors that rely on their assembly for survival (7,8). The quantification of epichaperome abundance serves as a functional analysis, acting as a biomarker for assessing proteome vulnerability to epichaperome disruption in patients' cells (8-11). This measurement provides a basis for patient selection in epichaperome therapy, offering a proteome-based rationale for precision medicine (12). To facilitate biomarker detection and quantification, a flow cytometry assay was developed (13). Using this assay, a patient was identified with high abundance of epichaperomes (AML patient with high epicherone abundance have hyperactivated signalosome) in the leukemic blasts predicting benefit from treatment with an epichaperome disruptor. The patient was treated under compassionate access with zelavespib, an epichaperome disruptor discovered by our team (7.) Zelavespib showed remarkable efficacy in this high-risk secondary AML patient with post-transplant relapse, with prolonged complete remission achieved without exacerbation of GvHD (11). This success could stem from a dual mechanism of zelavespib, first, a direct anti-tumor activity attributed to leukemia's vulnerability to epichaperome disruption and evidenced by the CR observed by day 45, and second an adjuvant effect, improving GvL (i.e., T cell anti-tumor efficacy) as evidenced by the sustained remission that lasted >5 years. This dual mechanism-which involves combating tumors directly and augmenting T cell function without exacerbating GvHD-would support the use of epichaperome disruptors in post-transplant settings. This also demonstrates the use of epichaperome assessment for medical treatment.

As the role of epichaperomes in T cell function was elucidated, the impact of epichaperome disruptors could be extended to other important applications such any T cell immunotherapy approach, that uses T cells such as CAR T cell therapies, where, despite the promising responses with FDA-approved products against CD19 in B-cell malignancies, approximately two thirds of patients still relapse (14-16).

The data demonstrate a role for the epichaperome in T cell activation, highlighting that disrupting the epichaperome significantly enhances T cell-mediated anti-tumor responses. These findings underscore the utility of epichaperome disruptors in post-transplant settings or in combination with T cell engagers or immune therapies like CAR-T cells. Harnessing the epichaperome in T cells thus represents a highly innovative approach, offering a novel combination treatment for cancer patients.

### Role of the Epichaperome in T Cells

Despite the evident importance of T cells in cancer therapy, the specific mechanisms sustaining and optimizing their activity remain largely unknown. This knowledge gap underscores the urgency and significance of this investigation into the contribution of the epichaperome to T cell dynamics, offering a novel perspective that provides novel avenues for enhancing cancer immunotherapy.

A flow cytometry assay was developed that allows for the precise assessment of epichaperome formation and its abundance at a single-cell level (7,13). Leveraging this assay, the dependencies of AML cells on the epichaperome is reported—with higher abundance proposing higher dependence—providing the ability to identify patients most likely to respond to epichaperome disruptors (7,17). Using this assay, an AML patient with high baseline epichaperomes in an AML patient transformed from MPN after failing allo-HSCT was identified. Treatment with zelavespib, an epichaperome disruptor (9), resulted in sustained CR for over 5 years (11). The remarkable response of this patient can also be attributed to the maintenance of donor T cell chimerism after the failed allo-HSCT, which led to the hypothesis that the epichaperome play a significant suppressive role in T cell function. Consequently, disrupting epichaperomes can revert these negative effects on T cells, and epichaperome disruptors improve anti-leukemia responses, particularly in the context of GvL.

The impact of epichaperomes may in part be manifested by skewing the balance of T cell populations, and/or by directly altering cellular processes that regulate T cell activity. During the activation process, naïve T cells differentiate into effector and memory T cells upon antigen recognition (21). Effector T cells can be activated by the same antigen to then be capable of eliminating the source of the antigen (for example, tumor specific antigen to eliminate a cancer cell) (21). The changes in cell immunophenotypic markers and cytokine production during the activation process are well-characterize (22). Epichaperome abundance can be characterized in distinct T cell subsets (23). Epichaperome formation and its abundance can be assessed using a flow cytometry assay (13) and then comparing unstimulated (resting) human T cells with T cells stimulated with anti-CD3/anti-CD28 antibody-coated microbeads (24). To this end, human peripheral blood mononuclear cells (PB-MNCs) will be obtained from healthy donors, T cells will be purified by positive selection of T cells using anti-CD4 and anti-CD8 magnetic beads (25). T cell activation will be performed in the presence of human recombinant IL-2 (rIL-2). The kinetics for the changes in surface molecules upon activation of T cells are well described and these surface markers can be used as a reference when assessing epichaperome abundance after activating the T cells (22). The assessment by flow cytometry can include the following antibodies to identify the activation/control of response markers and T cells subsets: CD45, CD3, CD4, CD8, CD25, CD69, CD40L, CD45RA, HLA-DR, CD38, CD122, CD132, and CD197 (CCR-7). A second panel can be used to identify T cell subsets with different functions such as Tregs, effector, naïve or memory T cells: CD3, CD4, CD8, CD45RA, CD45RO, CXCR4, CD62L, CD95, CD25, and CD127 (26). In addition, viability dyes can be included. Briefly, after adding activation beads, T cells can be sampled at different timepoints: 2 h, 4 h, 6 h, 12 h, 24 h, 48 h and 96 h; T cells can be labeled with the epichaperome probe PU-FITC or control-FITC and then stained with surface markers described above to distinguish different T cell subsets to assess epichaperome abundance (13). Epichaperome abundance can be measured relative to resting T cells. PD-1 and CTLA-4 can be expressed after 48 h from activation and play a role in the control of the activation response, thus these markers can also be also evaluated in the context of epichaperome abundance/function. As an orthogonal validation method for monitoring epichaperomes, the capillary isoelectric focusing immunoassays on NanoPro1000 can be used as previously reported (7). The NanoPro platform analyzes protein levels from minute samples (down to 25 cells) with high sensitivity (27) and retains proteins in a native configuration. Both the biochemical signature of the epichaperomes (i.e. stabilized complexes captured on native PAGE or IEF (7)) and the expression of other markers can be obtained using this method.

Epichaperome disruption in various biological contexts has been reported to normalize cellular protein networks (8, 29). This effect can manifest as cellular death in cancer cells or a reversal to a normal functioning state, as observed in cellular and mouse models of Alzheimer's disease (19), Parkinson's (30, 31). and traumatic brain injury (32). In the realm of T cell activation, epichaperome disruption may consequently rebalance protein networks, potentially leading to the facilitation of a specific cellular state defined by characteristic functions. In the multifaceted process of T cell activation, various components come into play, encompassing proliferation, effector function, and response control.

In vitro: a biased approach where the impact of the epichaperome disruptor PU-H71 is assessed on specific T cell subsets and functions, and an unbiased approach where transcriptional changes will be interrogated in T cells using single cell sequencing approaches will be undertaken. Purified CD3⁺ cells will be stimulated with anti-CD3/anti-CD28 antibody-coated microbeads and cultured in the presence of hIL-2 as described previously (33, 34). PU-H71 will be added (1) just before activation and (2) at the time point when epichaperomes are most abundant. Cell immunophenotype, viability, and proliferation will be assessed at 24 h, 48 h, and 96 h after activation using multiparameter flow cytometry. Two panels will be employed: one to identify the activation/control response markers and another to identify various T cell subsets with distinct functions such as Tregs, effector, naïve or memory T cells (CD3, CD4, CD8, CD45RA, CD45RO, CXCR4, CD62L, CD95, CD25, CD127, FoxP3), as described above. Annexin V/DAPI staining will be utilized to assess cell viability, and cell proliferation will be determined using carboxyfluorescein succinimidyl ester (CFSE) (35). scRNA seq will be performed to determine the overall changes in cell populations and the transcriptome of T cells exposed to PU-H71. Single-cell experiments will be performed on 10× Chromium platform according to established protocols and kits targeting 10K viable cells per experiment. Sequencing outputs will be processed by CellRanger (36) to generate counts matrices. Subsequent normalization, dimensionality reduction, clustering, and cellular annotation will be formed by established workflows such as OSCA (37) and Seurat (38). Results of the single-cell analysis will be distributed as R objects containing gene expression and cellular annotation and the source code will be distributed in open-source platforms (e.g. github) to ensure reproducibility.

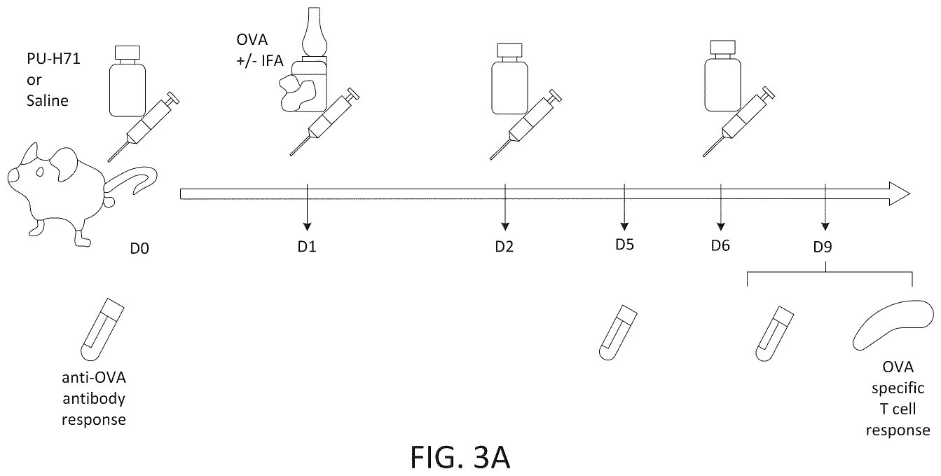

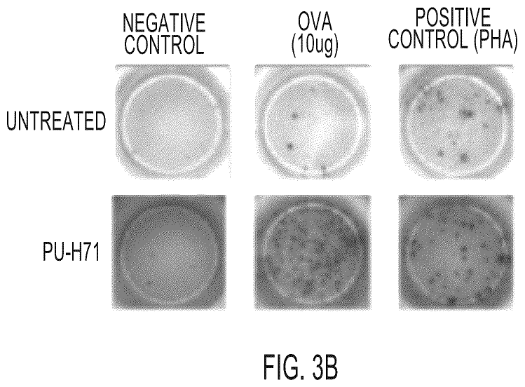

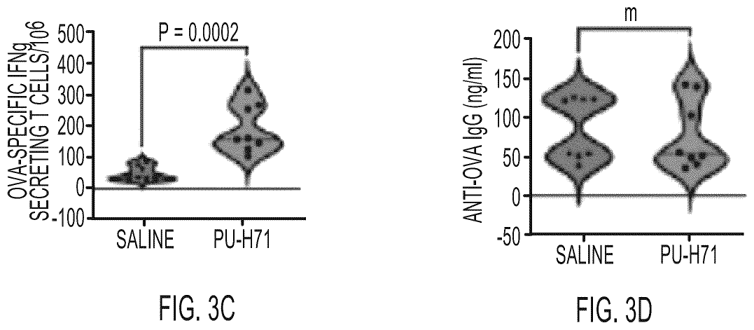

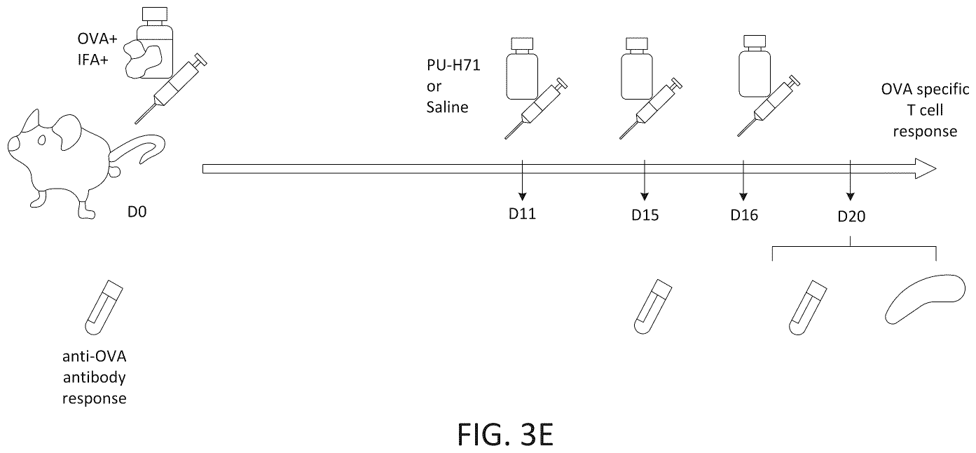

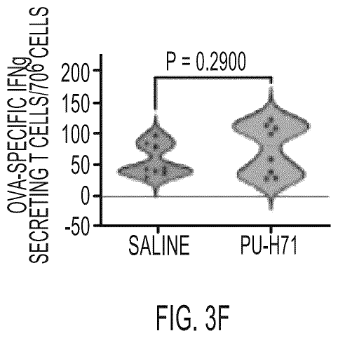

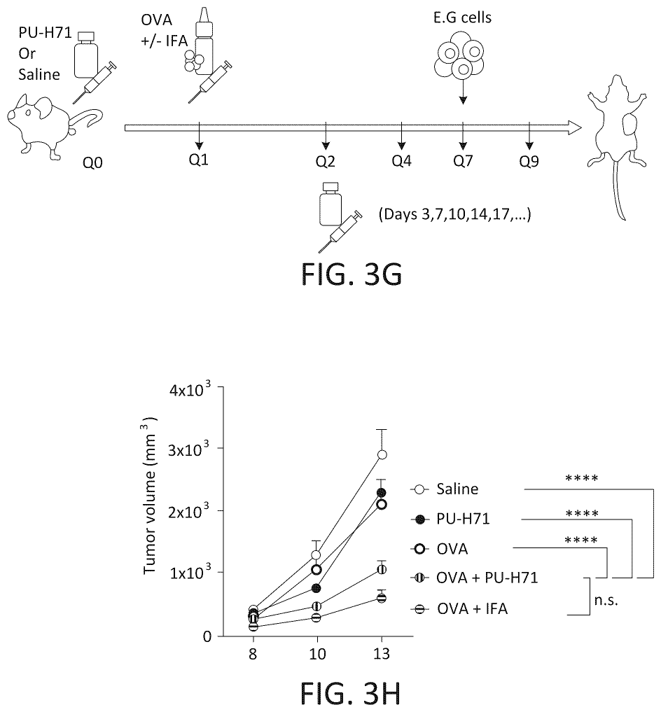

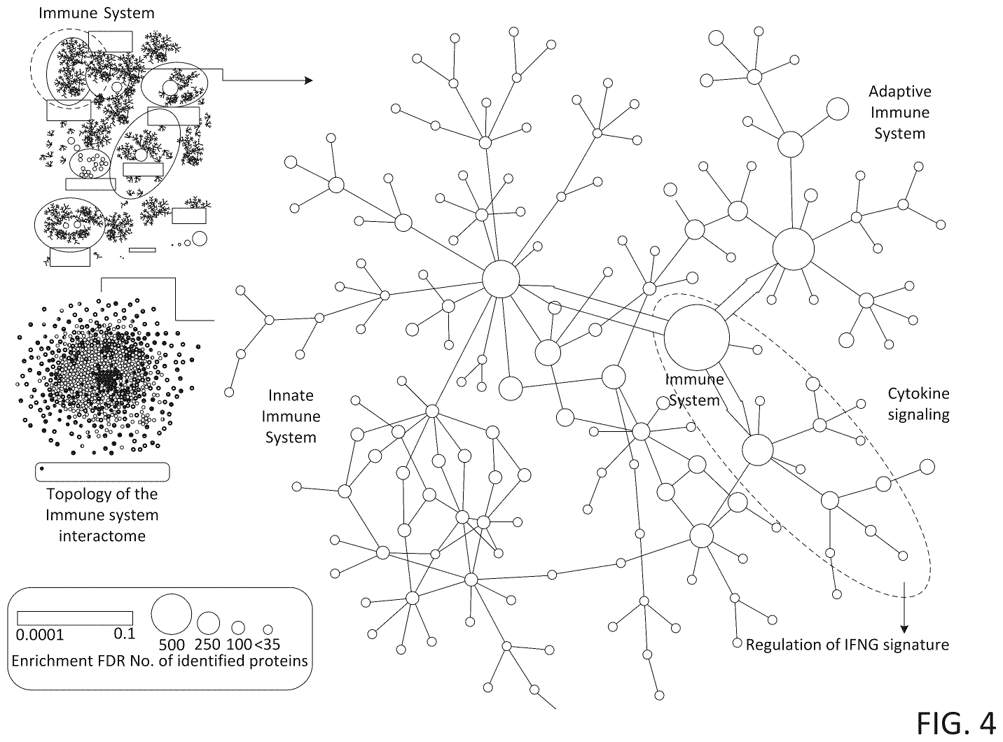

In vivo: In vivo assessment in immunization model: the well-characterized in vivo immunization model that utilizes chicken ovalbumin (OVA) will be used. The immune response of C57BL/6 mice to OVA is well characterized in the context of T cell responses and antibody production (39). Briefly, C57BL/6 mice (8 animals male and 8 female animals per cohort) will be injected subcutaneously (s.c.) with 100 μg OVA dissolved in PBS or emulsified in incomplete Freund's adjuvant (IFA) as positive control for the response. PU-H71 (at the target engaging dose of 75 mg/kg17) will be administered intraperitoneally (i.p) one day prior the treatment with OVA. Controls for the experiment will be C57BL/6 mice without OVA, and mice treated only with PU-H71. Mice will be sacrificed on day 9, and spleens will be harvested to evaluate OVA-specific T cell response using ELISPOT assays against IFNγ. Antibody production against OVA will be assessed in the peripheral blood (PB) at day 9 to confirm that the immunization took place (40). PU-H71 will be evaluated at different dosing schedules: before OVA administration to assess an effect on priming, shortly after OVA administration to assess an effect on T cell proliferation and polarization, and after the response has been completed (11 days) to evaluate an effect on the already ongoing response (for this cohort, mice will be sacrificed 9 days after PU-H71 treatment). Data (FIG. 3) shows that 8 mice per group (male and female) will allow for sufficient statistical power (>80% at two-sided alpha of 0.05) to detect differences of 1.6 standardized effect size in assessments and immune-cell characterization and quantification across groups.

In vivo assessment in an anti-tumor response model: E.G7 cells are OVA-expressing cells derived from the C57BL/6 (H-2b) mouse lymphoma cell line EL4, allowing for the establishment of a syngeneic lymphoma mouse model to evaluate anti-tumor protection after OVA immunization (39). More specifically, this model allows for the evaluation of MHC-I restricted responses of cytotoxic T lymphocytes in vivo. Mice (8 female and 8 male) will be first immunized with OVA (in the presence or absence of IFA (control) or PU-H71). At day 7 after immunization, lymphoma cells will be injected subcutaneously into the mice, and tumor growth will be evaluated twice a week using micro calipers. Tumor volume will be determined as mm³=length×width² (41). Survival will be also determined as an endpoint (mice will be euthanized when tumor size reaches 2000 mm³ or the animals lose more than 10% of weight). Differences in tumor growth and survival will be determined in mice immunized with OVA and PU-H71 compared to the controls OVA+IFA or no immunization. See FIG. 4.

Epichaperomes serve as scaffolding platforms with the capacity to rewire protein-protein interactions (PPIs) at a proteome-wide level (29, 44). This rewiring induces reorganization in thousands of proteins within cells, consequently influencing cellular phenotypes (8, 19). Thus, by understanding what proteins are impacted by epichaperomes, one can understand the functions of epichaperomes in the specific cellular context. To address the impact of epichaperomes on cellular networks in T cells, one can identify its interactors, and the cellular processes mediated by such interactors.

To investigate the epichaperome interactome, a mass spectrometry (MS)-based approach termed epichaperomics (8) can be used (see FIG. 4). This consists of an affinity purification method whereby an epichaperome bait (or a control bait) is incubated with homogenates (run in triplicate), and the recovered cargo is submitted for MS analysis and identification. The bait traps epichaperome-interactor assemblies enabling isolation of context-dependent epichaperome-proteome complexes from a given protein mixture (8, 19, 29, 45). This allows the epichaperome probes to be effective in capturing and retaining both direct and indirect interactors for analysis by MS. The cargo is subsequently subjected to interactor identification and quantification by MS using label-free data-independent acquisition (DIA) strategies, similar to methods previously reported (8, 19, 20). To examine the function of epichaperome interactors, pathway enrichment analyses can be performed.

To confirm interactor specificity, and validate their epichaperome-dependence, techniques complementary to affinity purification can be used, such as the cellular thermal shift assay (CETSA) (55). CETSA allows quantitative measurement of the stability of proteins and is based on the principle that heat-induced protein unfolding leads to rapid precipitation in the cellular environment. Epichaperome-interacting proteins would be prone to being influenced by epichaperome disruptors, such as PU-H71, in contrast to dispersed proteins in a cell. Proteins within stable complexes are more likely to co-aggregate upon heat denaturation and therefore epichaperome interactors would exhibit similar, non-random progressive insolubilities when subjected to an increasing temperature gradient (56). For this experiment, cells (or lysates) in triplicate are treated for 1.5 h (or as determined to be optimal) with PU-H71, and protein lysates are aliquoted and subjected to heat treatment, as we reported (8).

Flow cytometry assay was used to detect the epichaperomes in T cells from 10 healthy donors (5 female, 5 male). Samples were stained with CD45, CD3, CD4 and CD8. The MV4-11 AML cell line was used as a positive control for the test, as is of high epichaperome abundance and is epichaperome-dependent (7, 11, 17). It was found that, across all samples tested, in resting T cells epichaperome abundance was substantially and significantly below the established threshold for AML (FIG. 2A), consistent with a lack of epichaperome formation in resting T cells. Samples were assessed in triplicate and on independent days, and findings were also validated using samples after cryopreservation (data not shown). A study was performed to determine if epichaperomes form in T cells after activation (at a single time point, 20 h after activation). When T cells were activated via TCR using anti-CD3/anti-CD28 antibody-coated microbeads, epichaperome levels significantly increased (FIG. 2B), supporting a regulatory role of epichaperomes in T cell activity.

It was found that disrupting epichaperomes via PU-H71 had no detrimental effect on the viability of activated T cells, substantiating the notion that epichaperome formation in activated T cells supports processes other than cell survival (FIG. 2C). Interestingly, it was found that in the presence of PU-H71, the CD4 to CD8 ratio was skewed towards more CD8⁺ cells, while the expression of some activation markers such as CD69 were not significantly changed (FIG. 2D). Thus, epichaperome formation skews the balance of T cell populations and functions towards an immunosuppressive outcome, a balance that can be restored to a cytotoxic function by epichaperome disruptors. This leads to a role of epichaperome disruptors, such as PU-H71, in CAR T cell cytotoxicity and the ability of epichaperome disruptors, such as, PU-H71, to improve the outcomes of AML patients in the post-transplant setting by improving GvL without affecting GvHD.

For in vivo study, immune responses to OVA immunization were tested in C57BL/6 mice that were administered 75 mg/kg (3 doses, the first one prior OVA immunization) of PU-H71 (female mice; n=8; see FIG. 3A). OVA-specific T- and B-cell responses were evaluated by ELISPOT and ELISA, respectively. When administered at the time of priming, PU-H71 significantly increased the number of IFNγ-secreting T cells as compared to controls (median 126 vs 42 per million of cells, p=0.0002; n=8), without overt impact on T cell phenotype or anti-OVA antibody levels (FIG. 3A-D). In contrast, PU-H71 had no effect when administered once the OVA-specific T cell response was ongoing (FIG. 3E, F). This demonstrates that epichaperome disruption has a selective impact on IFNγ-secreting T cells, and its therapeutic implications may include targeted enhancement of cellular immune responses, provided the drug is administered at the appropriate time during the immune response.

Based on this observation, as study was designed to determine the ability of PU-H71 to improve anti-tumor T cell responses in which C57BL6 mice were immunized against OVA in the presence or the absence of PU-H71, and seven days later were challenged with E.G7, a mouse lymphoma cell line that expresses OVA. As a control, IFA was used to immunize the mice against OVA (FIG. 3G). OVA is a T cell-dependent antigen often used to study antigen-specific responses in mice (39, 40). Mice immunized against OVA in the absence of IFA, treated with PU-H71 had a significantly improved ability to control tumor progression as compared to mice immunized with OVA alone (18 days vs 16 days respectively; n=8, p=0.0192) or treated with PU-H71 without immunization (16 days; n=6, p=0.0027) or saline treatment (14 days; n=6 p=0.0009). FIG. 3H shows that OVA+PU-H71 significantly decreases tumor progression compared with OVA alone, PU-H71 alone, or saline treatment. The observed similarities in tumor progression between OVA+PU-H71 and the gold standard OVA+IFA highlight PU-H71's potential as an effective adjuvant in the anti-tumor T cell response. Even with an unoptimized dose and schedule, PU-H71 demonstrates comparable effectiveness to the current gold standard, OVA+IFA. These results demonstrate that PU-H71 has an adjuvant effect on the anti-tumor T cell response.

An epichaperome-abundant cancer cell line was used in further experiments. Epichaperomics clearly, and with high confidence, identified the epichaperome interactors that may be involved in immunosuppressive functions (FIG. 4). Pathway enrichment studies proposed that these proteins function in the following immunomodulatory protein networks: “Class I MHC mediated antigen processing and presentation”, “CTLA4 inhibitory signaling” and “PD-1 signaling”, “NFκB activity regulation” and “Regulation of interferon-gamma (IFNγ) signaling”. Taken together, these data suggest that epichaperomes support immune suppressive pathways.

Therapeutic Benefit of the Combination of an Epichaperome Disruptor with CAR T Cell Therapies

Chimeric antigen receptor T cell (CAR T) therapy has revolutionized hematological cancer treatment, particularly with FDA-approved CD19-targeted therapies for refractory large B-cell lymphoma (LBCL). Despite achieving over 80% overall response rates, a substantial challenge lies in the frequent relapse observed in approximately 60% of LBCL patients, with a limited median duration of response (11 months) (15, 16, 557). Thus, approaches are needed to improve the efficacy of CAR T therapy. Data provided herein demonstrate that epichaperome disruption results in improved anti-tumor T cell response in vaccine models, thus, it is believed that epichaperome disruption will improve the cytotoxic activity of CAR T cells by driving the T cell anti-tumor responses in addition to the CAR mediated killing.

The role of the epichaperome in CAR T cell activation was assessed, as well as the use of epichaperome disruptors in improving CAR T cell activity. It was determined whether the epichaperome exhibits similar activity in CAR T cells activated through target engagement or their TCR, as observed in healthy naïve T cells activated via their TCR. The impact of disrupting epichaperome formation on CAR T cell activity both in vitro and in vivo was investigated as well. To this end, CAR T cells targeting CD19 were used, as it is a line of therapy FDA-approved for CD19⁺ heme-malignancies, with human and mouse models. PU-H71 was used as an epichaperome disruptor.

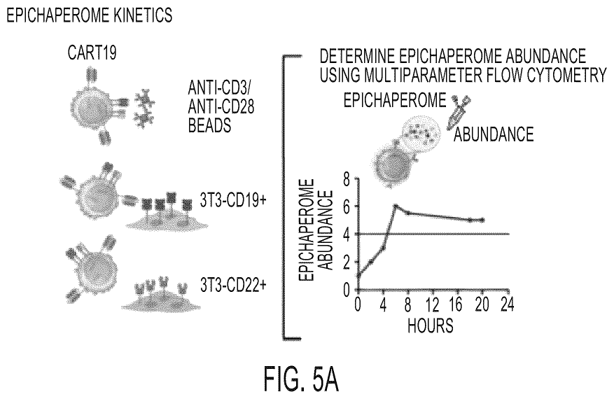

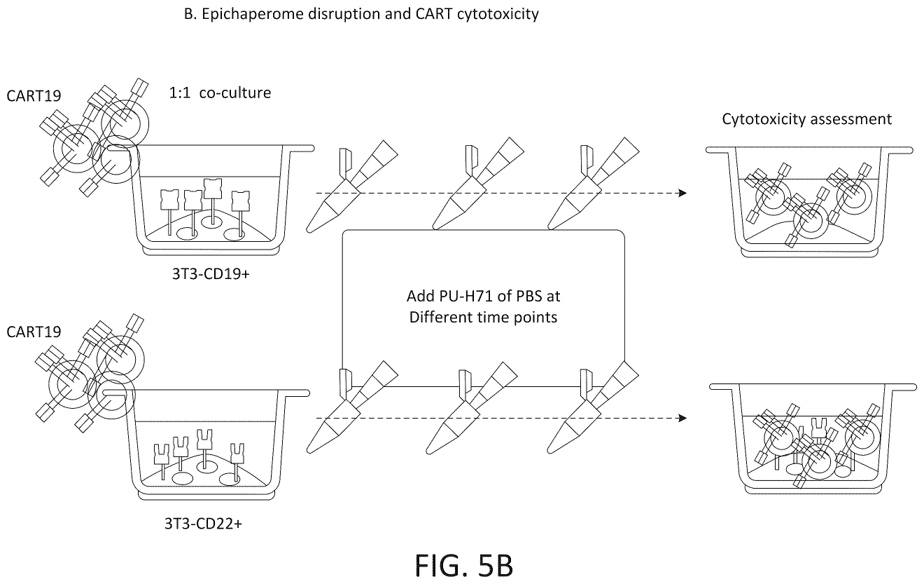

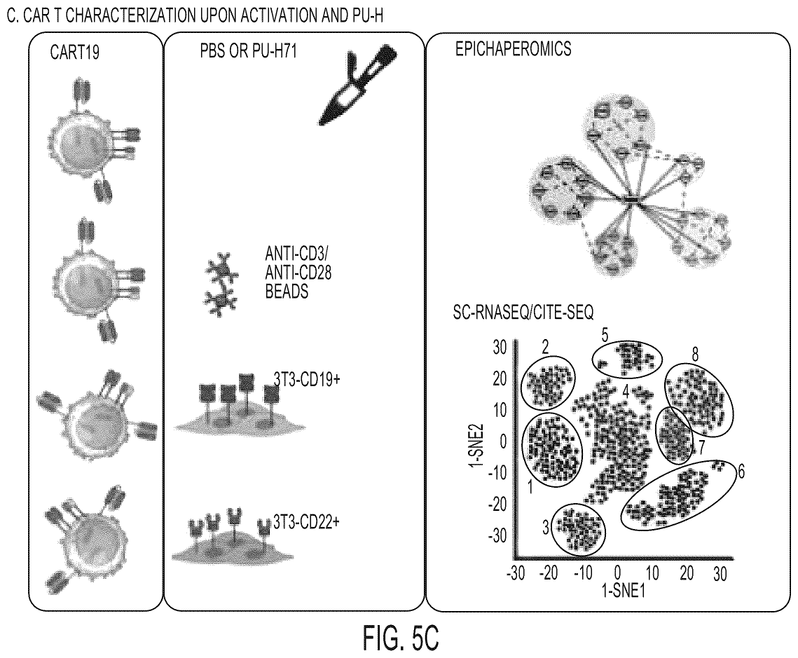

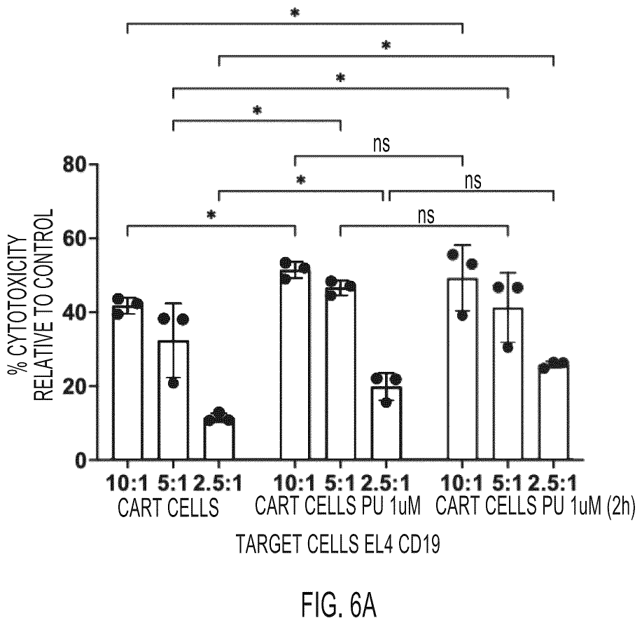

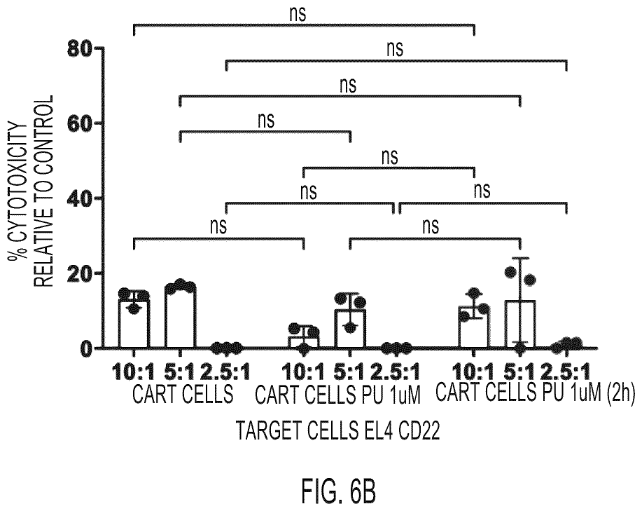

The impact of the epichaperome disruptors on CAR T cell activity can be assessed: (1) the kinetics of epichaperome formation, (2) the timing of epichaperome disruption for improvement of CAR T cell cytotoxicity and (3) mechanisms by which epichaperome regulate CAR T cells. (1) (see FIG. 5A) can use the flow cytometry assay (PU-FITC) to measure epichaperome abundance upon activation of CAR T cells via TCR (using anti-CD3/CD28 beads) and via target engagement (using 3T3 cells expressing human CD19, 373-CD19⁺; or expressing CD22, 373-CD22⁺ as a negative control) (58, 59). For this, CART19/4-1BB will be activated via TCR using anti-CD3/CD28 microbeads as described above, or by co-culturing 1:1 with 373-CD19⁺ or 373-CD22⁺ (control) cells, CART19/4-1BB will be sampled at different time points to evaluate epichaperome abundance. A murine adherent cell line transduced with human CD19 or CD22 can used to prevent alloreactivity (otherwise observed when using human target cells) while assessing CAR T cell function and facilitate the isolation of CART cells for transcriptional characterization (58). Following activation, the PU-FITC assay will be employed to assess the formation of the epichaperome in different T cell subpopulations at various time points. T cell subtypes will be distinguished using specific markers, including CD3, CD4, CD8, CD45RA, CD45RO, CXCR4, CD62L, CD95, CD25, CD 127, and FoxP3. This comprehensive will elucidate whether the kinetics of epichaperome assembly in CAR T cells, triggered via the TCR or target engagement, exhibit similarities. Additionally, similarities in the activation processes between CAR T cells and naïve T cells via the TCR may be seen. (2) (see FIG. 5B) will use 373-CD19⁺ as target cells, and 373-CD22⁺ cells as control (58). Co-cultures at suboptimal effector to target (E: T) ratios e.g. 0.5:1, 1:1, and 2.5:1 will be tested, PU-H71 (at concentrations with minimum to no effect on the tumor cells to be used, i.e. 0.5 μM and 1 μM) will be added at different time points starting at TO, 2 h, 4 h, 6 h, 8 h, 12 h, and 18 h. Overall cytotoxicity will be evaluated at 24 hours using flow cytometry (58, 59). (3) (see FIG. 5C) will compare activation via TCR and activation via epichaperome disruption. The evaluation will be performed as described above. The interactome of activated CAR T cells (via target and via TCR) will be compared with the interactome of activated T cells from described above.

To gain insights into the characteristics of CAR T cells post-exposure to PU-H71, cells activated through their TCR (anti-CD3/CD28 beads) or via CD19 target engagement will be assessed. Specifically, CART19/4-1BB cells will be treated with 1 μM PU-H71 at the time of activation (FIG. 5C). Cell collection will occur 6 h later for evaluation using single-cell RNA sequencing (scRNA-seq) with cellular indexing of transcriptomes and epitopes by sequencing (CITE-seq). The analysis will encompass T cell subset markers (CD4, CD8), activation markers (CD25, CD69, HLA-DR, CD38), effector/memory subsets (CD45RO, CD45RA, CD62L, CCR7), and checkpoints (PD-1, LAG3, CTLA4, TIGIT). The chosen 6 h analysis timepoint aligns with the data and existing datasets utilizing human anti-CD19 CART cells, allowing for meaningful comparisons (60). Additional time points can be used. Library preparation and sequencing will be carried out, as well as bioinformatic analysis.

Three mouse models that are often use to test CAR T cells in preclinical settings will be tested, the xenograft using human cell lines and human T cells (59, 61); a syngeneic model using mouse lymphoma with mouse CAR T cells in a immune competent setting, here one can model lymphodepletion as it is done in patients (62, 63), and a humanized setting utilizing patient samples and matching T cells and normal CD34+ cells (64).

Nalm-6 xenograft: Xenografts will be initiated using CD19⁺ B-cell acute lymphoblastic leukemia (B-ALL), Nalm-6 cells expressing luciferase and GFP (BLIV/GFP) transplanted into NSG mice (59). Anti-CD19 CAR T cells (CART19/4-1BB) or non-transduced T cells (NTC) will be administered once engraftment is confirmed by bioluminescence and flow cytometry. Four treatment cohorts will be studied: (1) saline, (2) PU-H71 (75 mg/kg i.p. bi-weekly), (3) CART19/4-1BB, (4) PU-H71 (75 mg/kgi.p. bi-weekly)+CART19/4-1BB. Each cohort will consist of 8 mice, ensuring >80% power at two-sided alpha of 0.05 to detect a standardized effect size of 1.6 in tumor burden and immune cell modulation, and will include both male and female mice. The PU-H71 dose and schedule will be determined based on data (9, 17). Tumor progression will be monitored weekly using bioluminescence imaging, with CAR T cell percentage and phenotype assessed in PB from day 2 onwards and every subsequent week, as published previously (17, 59). Bone marrow (BM) aspirates on day 14 will assess tumor burden and CAR T persistence. At the endpoint, the spleen, BM, and PB will be harvested to determine leukemia burden and CAR T cell phenotype.

Syngeneic B Cell Lymphoma Mouse Model: This model employs a functional immune system sensitive to mouse CAR T cells targeting CD19 and can be performed with a lymphodepletion regimen (62). In vitro, the effect of epichaperome inhibitor PU-H71 on mouse CAR T cells will be confirmed by assessing the A20 B cell lymphoma cell line for epichaperome abundance and sensitivity to PU-H71. Next, in vitro, cytotoxicity assays in the presence or absence of PU-H71 will be conducted with A20 cells and mouse CART19/28 (mCART19/28 cells). For in vivo assessment, BALB/C mice (H-2d) will be injected with A20/luciferase cells and distributed across four treatment cohorts: 1) saline, 2) PU-H71 (75 mg/kg i.p. bi-weekly), 3) CART19/28 (2×10⁶ cells), 4) PU-H71 (75 mg/kg i.p. bi-weekly)+CART19/28 (2×10⁶ cells). Each group, with both male and female mice, will include 8 animals, ensuring >80% power at two-sided alpha level of 0.05 to detect a standardized effect size of 1.6 in tumor burden and immune cell modulation. Tumor progression will be monitored through bioluminescence imaging, and mCAR T cell features and B cell aplasia will be assessed in PB starting on day 2 and weekly thereafter. At the study conclusion, the liver, lymph nodes, spleen, BM, and PB will be harvested to determine lymphoma burden and CAR T cell phenotype. Notably, mouse CD19 expression in relapsed animals will be evaluated to investigate antigen escape possibilities. If PU-H71-treated and mCART19/28-treated mice remain disease-free for over three weeks compared to control cohorts, surviving mice will undergo rechallenge with tumor cells. The presence and phenotype of CAR T cells will be assessed to confirm the efficacy of persistent CAR T cells. This model is already established in the Guzman lab (63).

ALL-Patient-Derived Xenografts in a Humanized Context: In this approach, T cell-depleted primary B-ALL cells will be co-injected with purified CD34⁺ cells from an HLA-A2 mismatched healthy donor to distinguish normal and malignant cells (64). T cells from the same healthy donor will be utilized to generate CART19/4-1BB cells based on the results of previous experiments. The anti-tumor effect will be evaluated by sampling PB and BM, along with profiling other immune compartments in the humanized PDX model. At the study's end, PB will be collected, and BM and spleen will be harvested to detect differences in tumor burden and immune cell modulation. Both male and female mice will be included in the evaluation. Epichaperome abundance will also be assessed in PB samples obtained two days after the initial treatment when T cells are detectable and expanding.

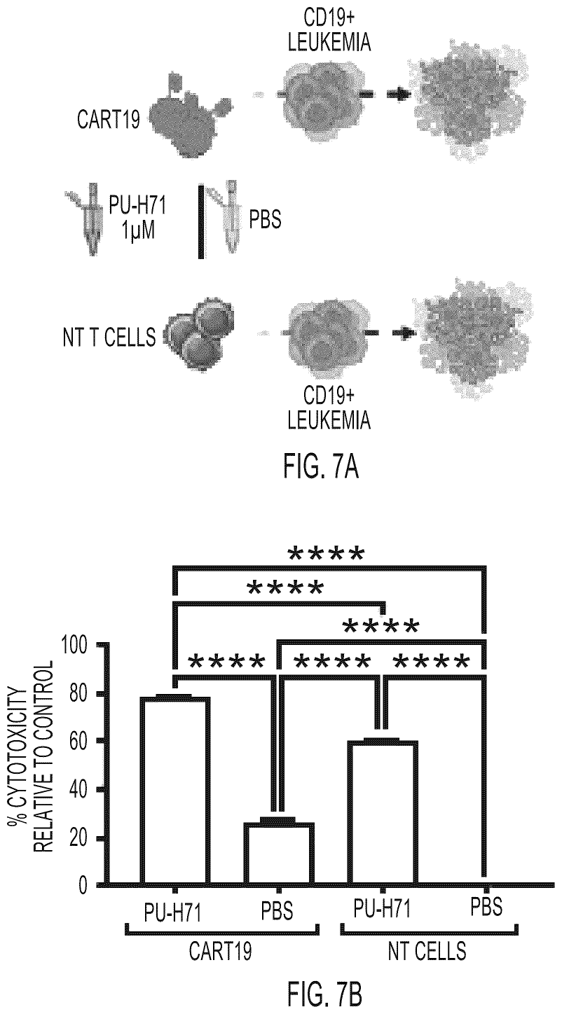

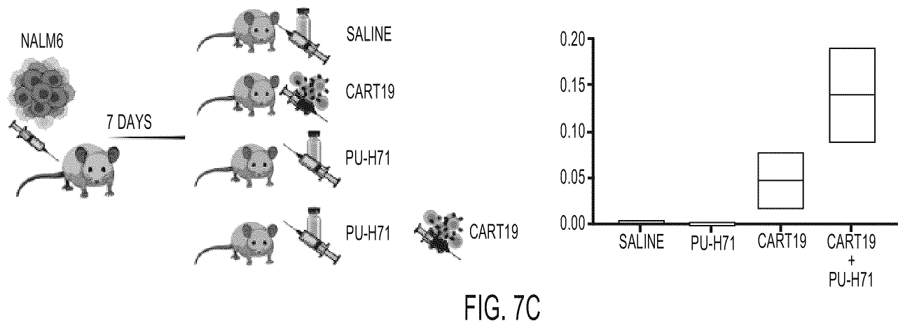

Epichaperome disruption using PU-H71 improves CAR T cell cytotoxic response. To investigate the impact of epichaperome disruption on CAR T cells, CART19/4-1BB was generated and tested. Similar to unstimulated T cells from healthy donors, a significant increase in epichaperome abundance was observed in activated CART19/4-1BB cells (not shown). Subsequently, the impact of epichaperome disruption on the function of CART19/4-1BB cells was assessed during co-culture with leukemia cells expressing CD19. The cytotoxic effect of CART19/4-1BB cells against Nalm-6 (CD19⁺ B-ALL cell line) was measured with or without 1 μM PU-H71 (FIG. 7). At an effector-to-target (E: T) ratio of 1:1, CART19/4-1BB cells in the presence of PU-H71 exhibited significantly higher cytotoxicity compared to co-cultures without PU-H71 (78% vs. 26%, respectively, p=0.002). Notably, control T cells (from the same healthy donor but not transduced with CART19/4-1BB) co-cultured with Nalm-6 in the presence of PU-H71 also displayed increased cytotoxicity against Nalm-6 (E: T ratio 1:1, 60% vs. 0%, p<0.0001). These results demonstrate that PU-H71 enhances the allogeneic cytotoxic function of T cells against Nalm-6 cells, and this effect is further potentiated by the presence of the CAR construct. In summary, these data show that disrupting the epichaperome represents a novel and feasible approach to improve CAR T cell therapy-leading to a more potent elimination of the tumor cells.

Effect of Epichaperome Disruption on Improving GvL without Increasing GvHD

The epichaperome disruptor, Zelavespib, as a single agent, induced a complete and sustained >5-year remission in an epichaperome-positive patient diagnosed with a MPN who progressed to AML, refractory to therapy, despite undergoing allo-HSCT (11). The patient-maintained donor T cell chimerism, despite the allo-HSCT failure, presenting atypical GvHD that was managed. Importantly, PU-H71 treatment did not exacerbate GvHD, underscoring its safety profile. The prolonged response observed in this patient strongly suggests that PU-H71's unique mechanism, potentially facilitating GvL, played a pivotal role. Data provided herein further supports this notion, demonstrating that epichaperome disruption enhances the anti-tumor response in a T cell-dependent OVA-expressing tumor model. Given that allo-HSCT remains the sole curative approach for AML and other hematologic malignancies, relying on GvL effects for efficacy, namely the ability of donor-derived T cells to eliminate host leukemia cell (66), it is hypothesized that PU-H71 treatment could improve allo-HSCT outcomes by specifically strengthening GvL effects, with little to no effect on GvHD. This unique potential positions PU-H71, as well as the other generations of epichaperome disruptors (8, 67, 68) as promising therapeutic agents in the landscape of hematologic malignancies and allo-HSCT.

Characterization of epichaperome disruption in vitro for GvL/GvHD. An established models for GvL/GvHD will be used (69-71). Specifically for the leukemia model component, P815 cells of DBA/2 (H-2d) mouse origin will be used. For the GvHD component, a hybrid B6×D2 F1 female recipients (8 to 12 weeks of age) will be used. First, the in vitro effect of PU-H71 on donor cells using a Mixed Lymphocyte-Tumor Reaction with P815 Cells will be assessed. Co-culture of BM and splenocytes from C57BL/6 (H-2b) as graft with irradiated splenocytes from B6×D2 F1 (H-2bxd) will be used. P815 tumor cells labeled with CFSE will be added to the coculture with or without PU-H71. P815 cells alone will be used as a control group. MLR will be kept for up to 4 days and viability of all cell types will be evaluated by flow cytometry.

Characterization of epichaperome disruption in vivo in a combined GvL/GvHD model. GvL/GvHD model will be established by transplanting lethally irradiated (10 Gy) B6×D2 F1 with 5×10⁶ T cells depleted BM (TDBM) cells from C57BL/6, 8.4×10⁶ to 9.2×10⁶ T cells from splenocytes, and 2×10⁶ malignant cells (i.v.) at the time of grafting. In another setting, 1·10⁶ malignant cells will be injected s.c. to evaluate the GvL effect against extramedullary leukemia. In this setting, one can expect to observe tumor growth around day 15. In all settings, PU-H71 will be injected i.p. the first week after transplantation every 3 days to evaluate its effect on GvHD and GvL just after transplantation. GvHD evaluation. Clinical score for GvHD will be evaluated twice a week as previously described (71) to evaluate the effect of PU-H71 on GvHD. Acute GvHD clinical score will be calculated based on the 5 following parameters: scored 0 (if absent) or 1 if present weight loss>10% of initial weight, hunching posture, skin lesion, dull fur, and diarrhea. Moribund/dead mice receive a total score of 5 (only if found dead after 10 days, which is likely due to lack of chimerism). Immunomonitoring. At different time points after transplantation (days 10, 20, and 30) the mice are euthanized and the spleen, liver, PB, and BM are collected for Immunomonitoring. Tail skin, lung, and gut are preserved in formaldehyde for GvHD evaluation by histology. T cells will be assessed by flow-cytometry using the following markers into two panels: CD3, CD4, CD25, CD62L, CD44, Foxp3, H-2Kb, H-2Kd, CD8, CTLA-4, TNFR2 and CD3, CD8, CD4, Foxp3, CD25, PD-1, Tbet, Eomes, TIM3, Grzb, H-2Kb, H-2Kd (70). Leukemia characterization. Leukemia cells will be monitored in the PB (weekly) and BM aspirates (every 3 weeks), the leukemia will be identified by the haplotype: Donors cells will be defined as H-2Kd^{negative} H-2Kb^{positive}, Recipient cells will be defined as H-2Kd^{positive} H-2Kb^{positive}, leukemia cells will be defined as H-2Kd^{positive} H-2Kb^{negative}. The abundance of the epichaperome in different mouse immune subsets: T cells: CD4⁺ T: CD3⁺ CD4⁺ CD8⁻ and CD8⁺ T: CD3⁺ CD4⁻ CD8⁺ Monocytes: classical: Ly6C⁺⁺, CD43⁺, intermediate: Ly6C⁺⁺ CD43⁺⁺, non-classical: Ly6C⁺, CD43⁺⁺⁷³ B cells: CD19⁺, CD20⁺ and NK cells: CD3-CD56⁺: CD3 NKp46⁺ or CD3-CD49b⁺ will also be tested. Control groups will be cohorts of irradiated mice receiving only TDBM, or TDBM and tumor cells. The TCR repertoire will be evaluated to better understand the impact of epichaperome inhibitors on T cell clonality using TCR sequencing by next-generation sequencing (74).

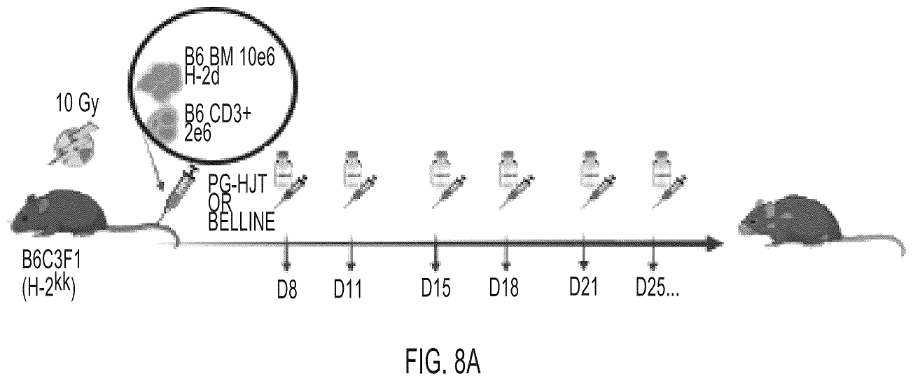

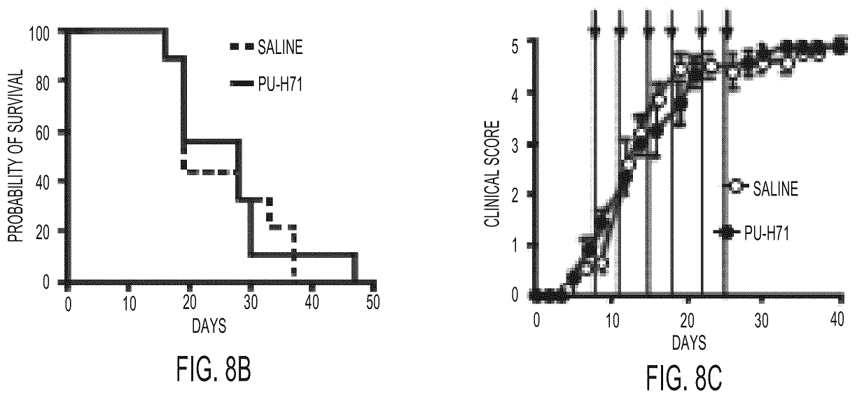

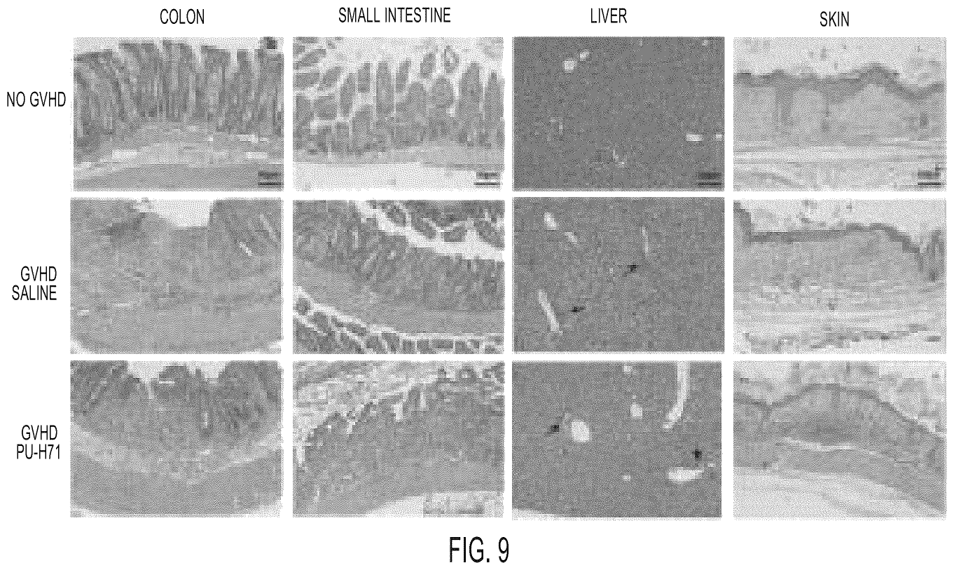

In a noteworthy case report detailed above, the patient, despite a failed allo-HSCT, maintained T cell chimerism that resulted in atypical GvHD. This GvHD manifestation was effectively managed with ruxolitinib (11). PU-H71 treatment did not exacerbate these symptoms, raising the intriguing possibility that PU-H71 might enhance GvL without inducing GvHD. Motivated by these observations, the impact of PU-H71 on GvHD was tested, utilizing an MHC mismatch mouse model (77). In this model, lethally irradiated B6×C₃ F1 (H-2b/k) mice were transplanted with bone marrow and splenocytes from C57BL/6 (H-2b) mice (FIG. 8). Mice were then divided into two cohorts: (1) PU-H71 75 mg/kg i.p. bi-weekly or (2) saline. Assessment included survival, GvHD clinical scores, and histological analysis of the colon, small intestine, liver, and skin. Encouragingly, the findings revealed that PU-H71 treatment in mice with low GvHD did not exacerbate the disease in terms of overall survival, clinical, or histological scores. These results provide reassurance regarding the safety profile of PU-H71 treatment during allo-HSCT.

### BIBLIOGRAPHY

1 Kantarjian, H. et al. Acute myeloid leukemia: current progress and future directions. Blood Cancer J 11, 41, doi:10.1038/s41408-021-00425-3 (2021).
2 de Lima, M., Roboz, G. J., Platzbecker, U., Craddock, C. & Ossenkoppele, G. AML and the art of remission maintenance. Blood Rev 49, 100829, doi:10.1016/j.blre.2021.100829 (2021).
3 Chiad, Z. & Chojecki, A. Graft versus Leukemia in 2023. Best Pract Res Clin Haematol 36, 101476, doi:10.1016/j.beha.2023.101476 (2023).
4 Sweeney, C. & Vyas, P. The Graft-Versus-Leukemia Effect in AML. Front Oncol 9, 1217, doi:10.3389/fonc.2019.01217 (2019).
5 Jagasia, M. et al. Risk factors for acute GVHD and survival after hematopoietic cell transplantation. Blood 119, 296-307, doi:10.1182/blood-2011-06-364265 (2012).
6 Di Ianni, M. et al. Cellular Strategies for Separating GvHD from GvL in Haploidentical Transplantation. Cells 13, doi:10.3390/cells13020134 (2024).
7 Rodina, A. et al. The epichaperome is an integrated chaperome network that facilitates tumour survival. Nature 538, 397-401, doi:10.1038/nature19807 (2016).
8 Rodina, A. et al. Systems-level analyses of protein-protein interaction network dysfunctions via epichaperomics identify cancer-specific mechanisms of stress adaptation. Nat Commun 14, 3742, doi:10.1038/s41467-023-39241-7 (2023).
9 Sharma, S. et al. Unraveling the Mechanism of Epichaperome Modulation by Zelavespib: Biochemical Insights on Target Occupancy and Extended Residence Time at the Site of Action. Biomedicines 11, doi:10.3390/biomedicines11102599 (2023).
10 Dunphy, M. P. S. et al. First-in-Human Trial of Epichaperome-Targeted PET in Patients with Cancer. Clin Cancer Res 26, 5178-5187, doi:10.1158/1078-0432.CCR-19-3704 (2020).
11 Sugita, M. et al. Targeting the epichaperome as an effective precision medicine approach in a novel PML-SYK fusion acute myeloid leukemia. NPJ Precis Oncol 5, 44, doi:10.1038/s41698-021-00183-2 (2021).
12 Pillarsetty, N. et al. Paradigms for Precision Medicine in Epichaperome Cancer Therapy. Cancer Cell 36, 559-573 e557, doi:10.1016/j.ccell.2019.09.007 (2019).
13 Merugu, S. et al. Chemical probes and methods for single-cell detection and quantification of epichaperomes in hematologic malignancies. Methods Enzymol 639, 289-311, doi:10.1016/bs.mie.2020.04.057 (2020).
14 June, C. H. & Sadelain, M. Chimeric Antigen Receptor Therapy. N Engl J Med 379, 64-73, doi:10.1056/NEJMra1706169 (2018).
15 Neelapu, S. S. et al. Axicabtagene Ciloleucel CAR T-Cell Therapy in Refractory Large B-Cell Lymphoma. N Engl J Med 377, 2531-2544, doi:10.1056/NEJMoa1707447 (2017).
16 Roschewski, M., Longo, D. L. & Wilson, W. H. CAR T-Cell Therapy for Large B-Cell Lymphoma-Who, When, and How? N Engl J Med 386, 692-696, doi:10.1056/NEJMe2118899 (2022).
17 Zong, H. et al. A Hyperactive Signalosome in Acute Myeloid Leukemia Drives Addiction to a Tumor-Specific Hsp90 Species. Cell Rep 13, 2159-2173, doi:10.1016/j.celrep.2015.10.073 (2015).
18 Taldone, T. et al. Synthesis of purine-scaffold fluorescent probes for heat shock protein 90 with use in flow cytometry and fluorescence microscopy. Bioorg Med Chem Lett 21, 5347-5352, doi:10.1016/j.bmcl.2011.07.026 (2011).
19 Inda, M. C. et al. The epichaperome is a mediator of toxic hippocampal stress and leads to protein connectivity-based dysfunction. Nat Commun 11, 319, doi:10.1038/s41467-019-14082-5 (2020).
20 Joshi, S. et al. Pharmacologically controlling protein-protein interactions through epichaperomes for therapeutic vulnerability in cancer. Commun Biol 4, 1333, doi:10.1038/s42003-021-02842-3 (2021).
21 Abbas, A. K. et al. Cellular and molecular immunology. Tenth edition. edn, (Elsevier, 2022).
22 Poloni, C. et al. T-cell activation-induced marker assays in health and disease. Immunol Cell Biol 101, 491-503, doi:10.1111/imcb. 12636 (2023).
23 Saxena, A., Dagur, P. K. & Biancotto, A. Multiparametric Flow Cytometry Analysis of Naive, Memory, and Effector T Cells. Methods Mol Biol 2032, 129-140, doi:10.1007/978-1-4939-9650-6_8 (2019).
24 Kalamasz, D. et al. Optimization of human T-cell expansion ex vivo using magnetic beads conjugated with anti-CD3 and Anti-CD28 antibodies. J Immunother 27, 405-418, doi:10.1097/00002371-200409000-00010 (2004).
25 Fuss, I. J. Purification of T Cell Populations. Curr Protoc Immunol 128, e94, doi:10.1002/cpim.94 (2020).
26 Mousset, C. M. et al. Comprehensive Phenotyping of T Cells Using Flow Cytometry. Cytometry A 95, 647-654, doi:10.1002/cyto.a.23724 (2019).
27 Padhan, N. et al. Highly sensitive and specific protein detection via combined capillary isoelectric focusing and proximity ligation. Sci Rep 7, 1490, doi:10.1038/s41598-017-01516-7 (2017).
28 Kaner, J. et al. Clonal Hematopoiesis and Premalignant Diseases. Cold Spring Harb Perspect Med 10, doi:10.1101/cshperspect.a035675 (2020).
29 Ginsberg, S. D., Sharma, S., Norton, L. & Chiosis, G. Targeting stressor-induced dysfunctions in protein-protein interaction networks via epichaperomes. Trends Pharmacol Sci 44, 20-33, doi:10.1016/j.tips.2022.10.006 (2023).
30 Kishinevsky, S. et al. HSP90-incorporating chaperome networks as biosensor for disease-related pathways in patient-specific midbrain dopamine neurons. Nat Commun 9, 4345, doi:10.1038/s41467-018-06486-6 (2018).
31 Ginsberg, S. D. et al. The penalty of stress-Epichaperomes negatively reshaping the brain in neurodegenerative disorders. J Neurochem 159, 958-979, doi:10.1111/jnc.15525 (2021).
32 Svirsky, S. E. et al. Experimental traumatic brain injury increases epichaperome formation. Neurobiol Dis 188, 106331, doi:10.1016/j.nbd.2023.106331 (2023).
33 Li, Y. & Kurlander, R. J. Comparison of anti-CD3 and anti-CD28-coated beads with soluble anti-CD3 for expanding human T cells: differing impact on CD8 T cell phenotype and responsiveness to restimulation. J Transl Med 8, 104, doi:10.1186/1479-5876-8-104 (2010).
34 Sattler, A. et al. Cytokine-induced human IFN-gamma-secreting effector-memory Th cells in chronic autoimmune inflammation. Blood 113, 1948-1956, doi:10.1182/blood-2008-02-139147 (2009).
35 Quah, B. J. & Parish, C. R. The use of carboxyfluorescein diacetate succinimidyl ester (CFSE) to monitor lymphocyte proliferation. J Vis Exp, doi:10.3791/2259 (2010).
36 Dempster, A. P., Laird, N. M. & Rubin, D. B. Maximum Likelihood from Incomplete Data via the EM Algorithm. Journal of the Royal Statistical Society. Series B (Methodological) 39, 1-38 (1977).
37 Amezquita, R. A. et al. Orchestrating single-cell analysis with Bioconductor. Nat Methods 17, 137-145, doi:10.1038/s41592-019-0654-x (2020).
38 Hao, Y. et al. Dictionary learning for integrative, multimodal and scalable single-cell analysis. Nature Biotechnology, doi:10.1038/s41587-023-01767-y (2023).
39 Fujinami, N. et al. Enhancement of antitumor effect by peptide vaccine therapy in combination with anti-CD4 antibody: Study in a murine model. Biochemistry and Biophysics Reports 5, 482-491, doi: https://doi.org/10.1016/j.bbrep.2016.02.010 (2016).
40 Littleton, L. C. & Gruenke, J. A. A comparison of the effect of three adjuvants on the antibody response to ovalbumin in mice. Bios 84, 142-147 (2013).
41 Fung, A. S., Lee, C., Yu, M. & Tannock, I. F. The effect of chemotherapeutic agents on tumor vasculature in subcutaneous and orthotopic human tumor xenografts. BMC Cancer 15, 112, doi:10.1186/s12885-015-1091-6 (2015).
42 Vasievich, E. A., Ramishetti, S., Zhang, Y. & Huang, L. Trp2 peptide vaccine adjuvanted with (R)-DOTAP inhibits tumor growth in an advanced melanoma model. Mol Pharm 9, 261-268, doi:10.1021/mp200350n (2012).
43 Viborg, N. et al. DNA based neoepitope vaccination induces tumor control in syngeneic mouse models. npj Vaccines 8, 77, doi:10.1038/s41541-023-00671-5 (2023).
44 Chiosis, G., Digwal, C. S., Trepel, J. B. & Neckers, L. Structural and functional complexity of HSP90 in cellular homeostasis and disease. Nat Rev Mol Cell Biol 24, 797-815, doi:10.1038/s41580-023-00640-9 (2023).
45 Ginsberg, S. D. et al. Disease-specific interactome alterations via epichaperomics: the case for Alzheimer's disease. FEBS J 289, 2047-2066, doi:10.1111/febs. 16031 (2022).
46 Mellacheruvu, D. et al. The CRAPome: a contaminant repository for affinity purification-mass spectrometry data. Nat Methods 10, 730-736, doi:10.1038/nmeth.2557 (2013).
47 Alam, Wangand, T. & Chiosis, G. Cytoscape files-Systems-level analyses of protein-protein interaction network dysfunctions via epichaperomics identify cancer-specific mechanisms of stress adaptation. Nature Communications. Zenodo, doi:10.5281/zenodo.7433980. (2022).
48 Wang, T., Digwal, C., Alam, A. & Chiosis, G. R Script Epichaperomics-Systems-level analyses of protein-protein interaction network dysfunctions via epichaperomics identify cancer-specific mechanisms of stress adaptation. Nature Communications. Zenodo., doi: https://doi.org/10.5281/zenodo.7416220 (2022).
49 Hermjakob, H. et al. IntAct: an open source molecular interaction database. Nucleic Acids Res 32, D452-455, doi:10.1093/nar/gkh052 (2004).
50 Stark, C. et al. BioGRID: a general repository for interaction datasets. Nucleic Acids Res 34, D535-539, doi:10.1093/nar/gkj 109 (2006).
51 Croft, D. et al. The Reactome pathway knowledgebase. Nucleic Acids Res 42, D472-477, doi:10.1093/nar/gkt1102 (2014).
52 Milacic, M. et al. Annotating cancer variants and anti-cancer therapeutics in reactome. Cancers (Basel) 4, 1180-1211, doi:10.3390/cancers4041180 (2012).
53 Kanehisa, M., Furumichi, M., Tanabe, M., Sato, Y. & Morishima, K. KEGG: new perspectives on genomes, pathways, diseases and drugs. Nucleic Acids Res 45, D353-D361, doi:10.1093/nar/gkw1092 (2017).
54 The Gene Ontology, C. Expansion of the Gene Ontology knowledgebase and resources. Nucleic Acids Res 45, D331-D338, doi:10.1093/nar/gkw1108 (2017).
55 Prabhu, N., Dai, L. & Nordlund, P. CETSA in integrated proteomics studies of cellular processes. Curr Opin Chem Biol 54, 54-62, doi:10.1016/j.cbpa.2019.11.004 (2020).
56 Tan, C. S. H. et al. Thermal proximity coaggregation for system-wide profiling of protein complex dynamics in cells. Science 359, 1170-1177, doi:10.1126/science.aan0346 (2018).
57 Boardman, A. P. & Salles, G. CAR T-cell therapy in large B cell lymphoma. Hematol Oncol 41 Suppl 1, 112-118, doi:10.1002/hon.3153 (2023).
58 Eugene-Norbert, M. et al. Development of optimized cytotoxicity assays for assessing the antitumor potential of CAR-T cells. J Immunol Methods 525, 113603, doi:10.1016/j.jim.2023.113603 (2024).
59 Sugita, M. et al. Radiation therapy improves CAR T cell activity in acute lymphoblastic leukemia. Cell Death Dis 14, 305, doi:10.1038/s41419-023-05829-6 (2023).
60 Bai, Z. et al. Single-cell antigen-specific landscape of CAR T infusion product identifies determinants of CD19-positive relapse in patients with ALL. Sci Adv 8, eabj2820, doi:10.1126/sciadv.abj2820 (2022).
61 Alhomoud, M., Martinet, J., Sugita, M., Gomez-Arteaga, A. & Guzman, M. L. Methods to monitor in vivo expansion and efficacy of CAR-T cells in preclinical models. Methods Cell Biol 167, 185-201, doi:10.1016/bs.mcb.2021.11.004 (2022).
62 Kuhn, N. F. et al. CD40 Ligand-Modified Chimeric Antigen Receptor T Cells Enhance Antitumor Function by Eliciting an Endogenous Antitumor Response. Cancer Cell 35, 473-488 e476, doi:10.1016/j.ccell.2019.02.006 (2019).
63 Alhomoud, M. et al. Low Dose Total Body Irradiation Improves CD19 CAR-T Expansion, Persistence, and Trafficking, Leading to Enhanced Efficacy in Large B-Cell Lymphoma. Blood 142, 1016-1016, doi:10.1182/blood-2023-185216 (2023).
64 Sugita, M. et al. Allogeneic TCRalphabeta deficient CAR T-cells targeting CD123 in acute myeloid leukemia. Nat Commun 13, 2227, doi:10.1038/s41467-022-29668-9 (2022).
65 Smith, R. & Shen, R. Complexities in comparing the impact of costimulatory domains on approved CD19 CAR functionality. J Transl Med 21, 515, doi:10.1186/s12967-023-04372-4 (2023).
66 Vincent, K., Roy, D. C. & Perreault, C. Next-generation leukemia immunotherapy. Blood 118, 2951-2959, doi:10.1182/blood-2011-04-350868 (2011).
67 Bolaender, A. et al. Chemical tools for epichaperome-mediated interactome dysfunctions of the central nervous system. Nat Commun 12, 4669, doi:10.1038/s41467-021-24821-2 (2021).
68 Yan, P. et al. Molecular Stressors Engender Protein Connectivity Dysfunction through Aberrant N-Glycosylation of a Chaperone. Cell Rep 31, 107840, doi:10.1016/j.celrep.2020.107840 (2020).
69 Lim, J. Y., Choi, M. S., Youn, H., Choi, E. Y. & Min, C. K. The influence of pretransplantation conditioning on graft-vs.-leukemia effect in mice. Exp Hematol 39, 1018-1029, doi:10.1016/j.exphem.2011.07.003 (2011).
70 Litvinova, E. et al. Graft-versus-leukemia effect after suicide-gene-mediated control of graft-versus-host disease. Blood 100, 2020-2025, doi:10.1182/blood-2002-01-0161 (2002).
71 Schmidt, F. et al. Flow cytometric analysis of the graft-versus-Leukemia-effect after hematopoietic stem cell transplantation in mice. Cytometry A 87, 334-345, doi:10.1002/cyto.a.22619 (2015).
72 Naserian, S. et al. Simple, Reproducible, and Efficient Clinical Grading System for Murine Models of Acute Graft-versus-Host Disease. Front Immunol 9, 10, doi:10.3389/fimmu.2018.00010 (2018).
73 Ziegler-Heitbrock, L. et al. Nomenclature of monocytes and dendritic cells in blood. Blood 116, e74-80, doi:10.1182/blood-2010-02-258558 (2010).
74 Dziubianau, M. et al. TCR repertoire analysis by next generation sequencing allows complex differential diagnosis of T cell-related pathology. Am J Transplant 13, 2842-2854, doi:10.1111/ajt.12431 (2013).
75 Choi, J. et al. In vivo administration of hypomethylating agents mitigate graft-versus-host disease without sacrificing graft-versus-leukemia. Blood 116, 129-139, doi:10.1182/blood-2009-12-257253 (2010).
76 Leclerc, M. et al. Control of GVHD by regulatory T cells depends on TNF produced by T cells and TNFR2 expressed by regulatory T cells. Blood 128, 1651-1659, doi:10.1182/blood-2016-02-700849 (2016).
77 Satake, A. et al. Separation of antileukemic effects from graft-versus-host disease in MHC-haploidentical murine bone marrow transplantation: participation of host immune cells. Int J Hematol 91, 485-497, doi:10.1007/s12185-010-0545-5 (2010).

Various embodiments of the invention are described above in the Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

What is claimed is:

1. A method of improving cytotoxicity of antigen-specific T cells in an animal, comprising:
administering an antigen to the animal to prime a T-cell response; and
administering an epichaperome disrupter to the animal during a priming phase with the antigen.

2. The method of claim 1, wherein administering the epichaperome disrupter to the animal concurrently with the antigen increases the number of IFNγ-secreting T cells compared to administering the antigen without the epichaperome disrupter.

3. The method of claim 1, wherein the animal is a human.

4. The method of claim 1, wherein the epichaperome disrupter is one or more compounds of Formula 1, 1A, 1B, 1C, 2, 3, 4, 5, 6, II, and/or III.

5. A method of improving cytotoxicity of antigen-specific T cells in an animal, comprising:
administering an immunotherapy to the animal; and
administering an epichaperome disruptor to the animal with the immunotherapy.

6. The method of claim 5, wherein the immunotherapy comprises a cellular immunotherapy.

7. The method of claim 6, wherein the cellular immunotherapy comprises chimeric antigen receptor (CAR)-T cell therapy or stem cell transplantation.

8. The method of claim 7, wherein the epichaperome disruptor is administered to the animal in an amount effective for improving the antitumor efficacy of the CAR-T cells.

9. The method of claim 7, wherein the epichaperome disruptor is administered to the animal in an amount effective for increasing GvL and decreasing GvHD.

10. The method of claim 5, wherein the animal is a human.

11. The method of claim 5, wherein the epichaperome disrupter is one or more compounds of Formula 1, 1A, 1B, 1C, 2, 3, 4, 5, 6, II, and/or III.

12. An antitumor vaccine for treating cancer in a human, the antitumor vaccine comprising: an adjuvant comprising an epichaperome disruptor; and a cellular immunotherapy.

13. The antitumor vaccine of claim 12, wherein the cellular immunotherapy comprises chimeric antigen receptor (CAR)-T cells (CAR-T cells) derived from the human's T-cells and modified to bind one or more antigens on a surface of cancer cells in the human.

14. The antitumor vaccine of claim 12, wherein the cellular immunotherapy comprises stem cell transplantation.

15. The antitumor vaccine of claim 12, further comprising an antigen.

16. The antitumor vaccine of claim 12, wherein the epichaperome disrupter is one or more compounds of Formula 1, 1A, 1B, 1C, 2, 3, 4, 5, 6, II, and/or III.

17. A vaccine for reducing or preventing infection in a human, the vaccine comprising: an adjuvant comprising an epichaperome disruptor; and an antigenic preparation or an an antigen-encoding RNA preparation.

18. The vaccine of claim 17, wherein the epichaperome disrupter is one or more compounds of Formula 1, 1A, 1B, 1C, 2, 3, 4, 5, 6, II, and/or III.
